SEQUENCE LISTING (9 sequences)

SEQ ID NO: 1 - protein, 364 aa, synthetic construct
  REGION 1..364: note = Synthetic
  VARIANT 1: note = optionally absent
  VARIANT 355..364: note = optionally absent
MSDEEERNELIKRIREAAQRAREAAERTGDPRVRELARELARIAQIAFYLVLHDPSSSEVNEALKAVVKAIELAVRALEEAEKTGDPEVRELAREVVRLAVEVATATAAGENDTLRKVAERALRLAKEAAKRGDAKAAKQAAKIAKLAAANAGDEDVLKKVELVRLAIELVEIVVENAKRKGDDDKEAAEAALAAFRIVLAAAQLAGIASLEVLELALRLIKEVVENAQREGYDIAVAAIAAAVAFAVVAVAAAAADITSSEVLELAIRLIKEVVENAQREGYVILLAALAAAAAFVVVAAAAKRAGITSSETLKRAIEEIRKRVEEAQREGNDISEAARQAAEEFRKKAEELKGSLEHHHHHH

SEQ ID NO: 2 - protein, 364 aa, synthetic construct
  REGION 1..364: note = Synthetic
  VARIANT 1: note = optionally absent
  VARIANT 355..364: note = optionally absent
MSDEEERNELIKRIREAAQRAREAAERTGDPRVRELARELARIAQIAFYLVLHDPSSSEVNEALKAVVKAIELAVRALEAAEKTGDPRVRELAREVVKAAVDVAEAAQAGLNDKLREVAEKALRLAKEALKEGDSTAAELAAEIARLAAKLAGDEDVLKKVKLVLEAIKLVKIVVENAKRKGDDSKEAAEAAVAAFLIVLAAAKLAGIASEEVLELAARLIKEVVENAQREGYDIAVAAIAAAVAFAVVAVAAAAADITSSEVLELAIRLIKEVVENAQREGYVILLAALAAAAAFVVVAAAAKRAGITSSETLKRAIEEIRKRVEEAQREGNDISEAARQAAEEFRKKAEELKGSLEHHHHHH

SEQ ID NO: 3 - protein, 381 aa, synthetic construct
  REGION 1..381: note = Synthetic
  VARIANT 1: note = optionally absent
  VARIANT 372..381: note = optionally absent
MSDEEERNELIKRIREAAQRAREAAERTGDPRVRELARELAKLAQIAFYLVLHDPSAKEVNLALELIVKAIELAVRALEEAEKTGDPHARELAREIVRLAVELARAVAEAAEEAKKQGNSELAEQVARAAQVALEVIKAAITAAKQGDRKAFRAALELVLEVIKAIEEAVKQGNPKKVAEVALKAELIRIVVQNAANKGDDADEAVEAARAAFEIVLAAAQLAGIDSEEVLELAARLIKEVVENAQREGYDIAVAAIAAAVAFAVVAVAAAAADITSSEVLELAIRLIKEVVENAVREGYVILLAALAAAAAFVVVAAAAKRAGITSSETLKRAIEEIRKRVEEAQREGNDISEAARQAAEEFRKKAEELKGSLEHHHHHH

SEQ ID NO: 4 - protein, 363 aa, synthetic construct
  REGION 1..363: note = Synthetic
  VARIANT 1: note = optionally absent
  VARIANT 354..363: note = optionally absent
MFNKSQQSAFYLILNMPNLNEAQRNGFIQSLKDDPAKSEVVAGEAAIEAARNASKKGSPETAREAVRLALELVQEAARVARKTGSTELLIAAAKLAIEVARVALKVGSPEAAREAVRAALELVQELIRAARKTGSKEVLEEAAKLALEVALVAAAVGSSEAAAKAVATAVEALKEAGASEDEIAEIVARVISEVIRILKENGSEYKVICVSVAKIVAEIVEALKRSGTSEDEIAEIVARVISEVIRTLKESGSDYLIICVCVAIIVAEIVEALKRSGTSEDEIAEIVARVISEVIRTLKESGSSYEVIKECVQIIVLAIILALMKSGTEVEEILLILLRVKTEVRRTLKESGSGSLEHHHHHH

SEQ ID NO: 5 - protein, 361 aa, synthetic construct
  REGION 1..361: note = Synthetic
  VARIANT 1: note = optionally absent
  VARIANT 352..361: note = optionally absent
MFNKSQQSAFYLILNMPNLNEAQRNGFIQSLKDDPSKSEVVAGEAAIEAARNALKKGSPETAREAVRLALELVQEAERQARKTGSTERLIAAAKLAIEVARVALKVGSPETAREAVRTALELVQELIRQARKTGSKEVLEEAAKLALEVAKVAAEVGSPETAARAVATAVEALKEAGASEDEIAEIVARVISEVIRILKESGSEYKVICRAVARIVAEIVEALKRSGTSEDEIAEIVARVISEVIRTLKESGSDYLIICVCVAIIVAEIVEALKRSGTSEDEIAEIVARVISEVIRTLKESGSSYEVIKECVQIIVLAIILALMKSGTEVEEILLILLRVKTEVRRTLKESGSLEHHHHHH

SEQ ID NO: 6 - protein, 314 aa, synthetic construct
  REGION 1..314: note = Synthetic
  VARIANT 1: note = optionally absent
  VARIANT 305..314: note = optionally absent
MFNKDQQSAFYEVLNMPNLNEAQRNGFIQSLKDDPSQSLKILIKAAAGGDSELEEVAKRIVKELAEQGRSEKEAAKEAAELIERITRAAGGNSDLIELAVRIVKILEEQGRSPSEAAKEAVEAIEAIVRAAGGDSEAIKVAAEIAKTIITQKESGSEYKEICRTVARIVAEIVEKLKRNGASEDEIAEIVAAIIAAVILTLKLSGSDYLIICVCVAIIVAEIVEALKRSGTSEDEIAEIVARVISAVIRVLKESGSSYEVIKECVQIIVLAIILALMKSGTEVEEILLILLRVKTEVRRTLKESGSLEHHHHHH

SEQ ID NO: 7 - protein, 422 aa, synthetic construct
  REGION 1..422: note = Synthetic
  VARIANT 1: note = optionally absent
  VARIANT 413..422: note = optionally absent
MFNKDQQSAFYEILNMPNLNEALRNGFIQLLKDDPSKSTVILTAAKVAAELSEKIRTLKESGSSYEQIAETVAKAVAKLVEKLKRNGVSEDEIALAVALIISAVIQTLKESGSSYEVIAEIVARIVAEIVEALKRSGTSEDEIAEIVARVISEVIRTLKESGSSYEVIAEIVARIVAEIVEALKRSGTSEDEIAKIVARVIAEVLRTLKESGSSEEVIKEIVARIITEIKEALKRSGTSEDEIELITLMIEAALEIAKLKSSGSEYEEICEDVARRIAELVEKLKRDGTSAVEIAKIVAAIISAVIAMLKASGSSYEVICECVARIVAEIVEALKRSGTSAAIIALIVALVISEVIRTLKESGSSFEVILECVIRIVLEIIEALKRSGTSEQDVMLIVMAVLLVVLATLQLSGSLEHHHHHH

SEQ ID NO: 8 - protein, 355 aa, synthetic construct
  REGION 1..355: note = Synthetic
  VARIANT 1: note = optionally absent
  VARIANT 346..355: note = optionally absent
MSDEEERNELIKRIREAAQRAREAAERTGDPRVRELARELARLAQRAFYLVLHDPSSSDVNEALKLIVEAIEAAVRALEAAERAGDPELREDAREAVRLAVEAAEEVQRNPSSSTANLLLKAIVALAEALAAAANGDKEKFKKAAESALEIAKRVVEVASKEGDPEAVLEAAKVALRVAELAAKNGDKEVFKKAAESALEVAKRLVEVASKEGDPELVLEAAKVALRVAELAAKNGDKEVFQKAAASAVEVALRLTEVASKEGDSELETEAAKVITRVRELASKQGDAAVAILAETAEVKLEIEESKKRPQSESAKNLILIMQLLINQIRLLVLQIRMLDEQRQEGSLEHHHHHH

SEQ ID NO: 9 - protein, 364 aa, synthetic construct
  REGION 1..364: note = Synthetic
  VARIANT 1: note = optionally absent
  VARIANT 355..364: note = optionally absent
MSDEEERNELIKRIREAAQRAREAAERTGDPRVRELARELARLAQRAFYLVLHDPSSSDVNEALKLIVEAIEAAVRALEAAERTGDPKVREEARELVRRAVEAAEEVQRNPSSSEVNEKLKAIVVEIEVKVASLEAKEVTDPDKALKIAKKVIELALEAVKENPSTEALRAVLEAVRLASEVAKRVTDPDKALKIAKLVIELALEAVKEDPSTDALRAVLEAVRLASEVAKRVTDPDKALKIAKLVLELAAEAVKEDPSTDALRAAKEAERLATEVAKRVTDPKKAREIEMLVLKLQMEAILAETEEVKKEIEESKKRPQSESAKNLILIMQLLINQIRLLALQIRMLALQLQEGSLEHHHHHH

(19) United States
(12) Patent Application Publication
(10) Pub. No.: US 2025/0049731 A1
(43) Pub. Date: Feb. 13, 2025

(54) DESIGNED ANTIBODY-BOUND NANOPARTICLES

(71) Applicant: University of Washington, Seattle, WA (US)

(72) Inventors: Robert Divine, Seattle, WA (US); Ivan Vulovic, Seattle, WA (US); David Baker, Seattle, WA (US); William H. Sheffler, Seattle, WA (US); Franziska Seeger, Seattle, WA (US)

(21) Appl. No.: 18/912,238

(22) Filed: Oct. 10, 2024

Related U.S. Application Data
(63) Continuation of application No. 17/907,372, filed on Sep. 26, 2022.
(60) Provisional application No. 63/085,351, filed on Sep. 30, 2020.
(60) Provisional application No. 63/036,062, filed on Jun. 8, 2020.

(51) Int. Cl. A61K 9/51; C07K 14/32; C07K 16/28

(52) U.S. Cl. CPC A61K 9/5169; C07K 14/32; C07K 16/2878; C07K 2317/92; C07K 2319/30; C07K 2319/70

(57) ABSTRACT

Polypeptides are disclosed comprising an (Fc) binding domain, a helical polypeptide monomer, and an oligomer domain, polymers thereof, and uses thereof.

### SEQUENCE LISTING STATEMENT

A computer readable form of the Sequence Listing is filed with this application by electronic submission and is incorporated into this application by reference in its entirety. The Sequence Listing is contained in the file created on Sep. 20, 2022 having the file name “20-860-WO-US.xml” and is 22 kb in size.

### BACKGROUND

Antibodies are very widely used in therapeutics and diagnostics applications. While there have been some efforts to oligomerize antibodies to enhance avidity and receptor clustering, there are no current methods to precisely form ordered and structurally homogeneous antibody-bound nanoparticle structures.

### SUMMARY

In one aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-9, wherein residues in parentheses are optional (i.e.: not considered in the percent identity requirement), wherein the polypeptide is capable of (a) assembling into a polymer, including but not limited to a homo-polymer, and (b) binding to a constant region of an IgG antibody.

In other aspects, the disclosure provides nucleic acid encoding the polypeptide of any embodiment of the disclosure, expression vectors comprising the nucleic acids of the disclosure operatively linked to a control sequence, and host cells comprising the polypeptide, nucleic acid, and/or expression vector of any embodiment herein.

In another aspect, the disclosure provides polymers of the polypeptide of embodiment of the disclosure, wherein
(i) each monomer in the polymer comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:1;
(ii) each monomer in the polymer comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:2;
(iii) each monomer in the polymer comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:3;
(iv) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:4;
(v) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:5;
(vi) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:6;
(vii) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:7;
(viii) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:8; or
(ix) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:9;
wherein residues in parentheses are optional (i.e.: not considered in the percent identity requirement).

In another aspect, the disclosure provides particles, comprising:
(a) a plurality of identical polymers according to any embodiment herein, and
(b) a plurality of antibodies comprising Fc domains;
wherein
   (i) each antibody in the plurality of antibodies comprises a first Fc domain and a second Fc domain;
   (ii) each antibody in the plurality of antibodies is (A) non-covalently bound via the first Fc domain to one polypeptide monomer chain of a first homo-polymer, and (B) non-covalently bound via the second Fc domain to one polypeptide monomer of a second homo-polymer; and
   (iii) each polypeptide monomer chain of each homo-polymer is non-covalently bound to one Fc domain;
wherein the particle comprises dihedral, tetrahedral, octahedral, or icosahedral symmetry.

In one aspect, the disclosure provides particles, comprising:
(a) a plurality of polypeptide polymers, wherein
   (i) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:1;
   (ii) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:2;
   (iii) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:3;
   (iv) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:4;
   (v) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:5;
   (vi) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:6;
   (vii) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:7;
   (viii) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:8; or
   (ix) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:9; wherein residues in parentheses are optional (i.e.: not considered in the percent identity requirement); and
(b) a plurality of antibodies comprising Fc domains, wherein
   (i) each antibody in the plurality of antibodies comprises a first Fc domain and a second Fc domain;
   (ii) each antibody in the plurality of antibodies is (A) non-covalently bound via the first Fc domain to one polypeptide monomer chain of a first polymer, and (B) non-covalently bound via the second Fc domain to one polypeptide monomer of a second polymer; and
   (iii) each polypeptide monomer chain of each polymer is non-covalently bound to one Fc domain;
wherein the particle comprises dihedral, tetrahedral, octahedral, or icosahedral symmetry.

In other aspects, the disclosure provides compositions comprising a plurality of the particles of any embodiment of the disclosure; pharmaceutical compositions comprising (a) the polypeptides, polymers, particles, or compositions of any embodiment herein, and (b) a pharmaceutically acceptable carrier; methods for using the polypeptides, nucleic acids, expression vectors, host cells, polymers, particles, compositions, or pharmaceutical compositions for any suitable use, including but not limited to those described in the examples, and including for the diagnostic or therapeutic use of antibodies present in the particles and compositions; and polypeptide computational design methods as disclosed in the examples.

### DESCRIPTION OF THE FIGURES

FIG. 1. Antibody nanocage (AbC) design. A, Polyhedral geometry is specified. B, An antibody Fc model from hIgG1 is aligned to one of the C2 axes (in this case, a D2 dihedron is shown). C, Antibody Fc-binders are fused to helical repeat proteins that are then fused to the monomeric subunit of helical cyclic oligomers. All combinations of building blocks and building block junctions are sampled (below inset). D-E, Tripartite fusions that successfully place the cyclic oligomer axis in the orientation required for the desired polyhedral geometry (D) move forward for sidechain redesign (E). F, Designed AbC-forming oligomers are bacterially expressed, purified, and assembled with antibody Fc or IgG.

FIG. 2. Structural characterization of AbCs. A, Design models, with antibody Fc and designed AbC-forming oligomers. B, Overlay of SEC traces of assembly formed by mixing design and Fc with those of the single components. C, EM images with 2D averages in inset; all data is from negative-stain EM with the exception of designs o42.1 and i52.3 (cryo-EM). D-E, SEC (D) and NS-EM representative micrographs with 2D class averages (E) of the same designed antibody cages assembled with full human IgG1 (with the 2 Fab regions intact).

FIG. 3. 3-Dimensional reconstructions of AbCs formed with Fc. Computational design models (cartoon representation) of each AbC are fit into the experimentally-determined 3D density from EM. Each nanocage is viewed along an unoccupied symmetry axis (left), and after rotation to look down one of the C2 axes of symmetry occupied by the Fc (right). 3D reconstructions from o42.1 and i52.3 are from cryo-EM analysis; all others, from NS-EM.

FIG. 4. AbCs activate apoptosis and angiogenesis signaling pathways. (A and B) Caspase-3/7 is activated by AbCs formed with α-DR5 antibody (A), but not the free antibody, in RCC4 renal cancer cells (B). (C and D) α-DR5 AbCs (C), but not Fc AbC controls (D), reduce cell viability 4 days after treatment. (E)-DR5 AbCs reduce viability 6days after treatment. (F and G) o42.1 a-DR5 AbCs enhance PARP cleavage, a marker of apoptotic signaling; (G) is a quantification of (F) relative to PBS control. (H) The F-domain from angiopoietin-1 was fused to Fc (A1F-Fc) and assembled into octahedral (o42.1) and icosahedral (i52.3) AbCs. (I) Representative Western blots show that A1F-Fc AbCs, but not controls, increase pAKT and pERK1/2 signals. (J) Quantification of (I): pAKT quantification is normalized to o42.1 A1F-Fc signaling (no pAKT signal in the PBS control); pERK1/2 is normalized to PBS. (K) A1F-Fc AbCs increase vascular stability after 72 hours. (Left) Quantification of vascular stability compared with PBS. (Right) Representative images; scale bars, 100 mm. All error bars represent means±SEM; means were compared using analysis of variance and Dunnett post-hoc tests (tables 11 and 12). *P≤0.05; **P≤0.01; ***P≤0.001; ****P≤0.0001.

FIG. 5, α-CD40 AbCs activate CD40 signaling over uncaged IgGs. A-D, Octahedral AbCs produced with α-CD40) (A) form AbCs of the expected size and shape according to SEC (B), DLS (C), and NS-EM (D). E, CD40 pathways are activated by LOB7/6 α-CD40) octahedral nanocages but not by free LOB7/6. Scale bars represent means±SD, n=3; EC50s reported in Table 7.

FIG. 6. Designed Fc-binding designed helical repeat. A, Model of the helical repeat protein DHR79 docked against antibody Fc (PDB ID: 1DEE). Residues from protein A (PDB ID: 1L6X) are grafted at the interface between the Fc and the helical repeat protein. B, SEC trace of the Fc-binding helical repeat monomer. C, Biolayer interferometry (BLI) of the Fc-binding helical repeat design with Fc (left) or with hIgG1 (right), with summary statistics (below).

FIG. 7. Additional α-DR5 AbC experiments. A, α-DR5 AbCs and TRAIL activate caspase-3,7 in Colo205 colorectal cancer cell lines. B-C, AbCs formed with Fc from hIgG1 do not activate caspase-3,7 (B) or reduce viability (C) in RCC4 cells. D, α-DR5 AbCs do not greatly activate caspase-3,7 after 2 d (D) or reduce viability (E) in a primary tubular kidney cell line (RAM009). F, Cleaved PARP is activated by α-DR5 in RCC4 cells, but not by TRAIL, α-DR5, or Fc AbCs.

FIG. 8. Additional A1F-Fc AbC experiments. A-B, o42.1 and i52.3 AbCs formed with A1F-Fc are monodisperse and of the expected size per SEC on a Superose™ 6 column (A) and DLS (B). SEC shows the assembly trace in black, the relevant AbC design component in light grey, and the A1F-Fc in dark grey. C. A control assembly displaying 8 A1F ligands (“H8-A1F”) produced similar levels of pAKT and pERK1/2 activation to A1F-Fc AbCs along with a comparable increase in vascular stability; data for all other conditions besides H8-A1F are replotted for convenience from FIG. 4i-k. D. Representative images of o42.1, i52.3 AbCs, and H8-A1F formed with Fc in the vascular stability assays; scale bars are 100 μm. E, o42.1 A1F-Fc AbCs were incubated with 100% human serum (HS) for 24 hours at 4° C. or 37° C. and applied to HUVEC cells at 150 nM. pAKT signal showed no decrease from o42.1 A1F-Fc particles incubated with serum. Statistical analyses are reported in Table 12.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M.P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R.I. Freshney. 1987. Liss, Inc. New York, NY), Gene Transfer and Expression Protocols, pp. 109-128, ed. E.J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In all embodiments of polypeptides disclosed herein, any N-terminal methionine residues are optional (i.e.: the N-terminal methionine residue may be present or may be absent).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In a first aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-9, wherein residues in parentheses are optional (i.e.: not considered in the percent identity requirement), wherein the polypeptide is capable of (a) assembling into a polymer, including but not limited to a homo-polymer, and (b) binding to a constant region of an IgG antibody.

TABLE 1
Sequences and original building blocks used for all designs.

| | Fc | Monomer | Oligomer | |
|---|---|---|---|---|
| | binder | (standard | (bold | |
| Name | (underlined) | font) | font) | Sequences |
| d2.3 | DHR79 | EXT6- | bex2c2_G2 | (M)SDEEERNELIKRIREAAQRAREAAERTGDPRVRELARELARIAQIAFYLVLH |
| SEQ ID | | DHR62 | | DPSSSEVNEALKAVVKAIELAVRALEEAEKTGDPEVRELAREVVRLAVEVATATA |
| NO: 1 | | | | AGENDTLRKVAERALRLAKEAAKRGDAKAAKQAAKIAKLAAANAGDEDVLKKVEL |
| | | | | VRLAIELVEIVVENAKRKGDDDKEAAEAALAAFRIVLAAAQLAGIASLEVLELAL |
| | | | | RLIKEVVENAQREGYDIAVAAIAAAVAFAVVAVAAAAADITSSEVLELAIRLIKE |
| | | | | VVENAQREGYVILLAALAAAAAFVVVAAAAKRAGITSSETLKRAIEEIRKRVEEA |
| | | | | QREGNDISEAARQAAEEFRKKAEELK(GSLEHHHHHH) |
| d2.4 | DHR79 | EXT6- | bex2c2_G2 | (M)SDESERNELIKRIREAAQRAREAAERTGDPRVRELARELARIAQIAFYLVLH |
| SEQ ID | | DHR62 | | DPSSSEVNEALKAVVKAIELAVRALEAAEKTGDPRVRELAREVVKAAVDVAEAAQ |
| NO: 2 | | | | AGLNDKLREVAEKALRLAKEALKEGDSTAAELAAEIARLAAKLAGDEDVLKKVKL |
| | | | | VLEAIKLVKIVVENAKRKGDDSKEAAEAAVAAFLIVLAAAKLAGIASEEVLELAA |
| | | | | RLIKEVVENAQREGYDIAVAAIAAAVAFAVVAVAAAAADITSSEVLELAIRLIKE |
| | | | | VVENAQREGYVILLAALAAAAAFVVVAAAAKRAGITSSETLKRAIEEIRKRVEEA |
| | | | | QREGNDISEAARQAAEEFRKKAEELK(GSLEHHHHHH) |
| d2.7 | DHR79 | DHR76 | bex2c2_G3 | (M)SDEEERNELIKRIREAAQRAREAAERTGDPRVRELARELAKLAQIAFYLVLH |
| SEQ ID | | | | DPSAKEVNLALELIVKAIELAVRALEEAEKTGDPHARELAREIVRLAVELARAVA |
| NO: 3 | | | | EAAEEKKQGNSELAEQVARAAQVALEVIKAAITAAKQGDRKAFRAALELVLEVI |
| | | | | KAIEEAVKQGNPKKVAEVALKAELIRIVVQNAANKGDDADEAVEAARAAFEIVLA |
| | | | | AAQLAGIDSEEVLELAARLIKEVVENAQREGYDIAVAAIAAAVAFAVVAVAAAAA |
| | | | | DITSSEVLELAIRLIKEVVENAVREGYVILLAALAAAAAFVVVAAAAKRAGITSS |
| | | | | ETLKRAIEEIRKRVEEAQREGNDISEAARQAAEEFRKKAEELK(GSLEHHHHHH) |
| t32.4_old | Protela A | EXT6- | tj04c3_int5v2 | (M)FNKSQQSAFYLILNMPNLNEAQRNGFIQSLKDDPAKSEVVAGEAAIEAARNA |
| SEQ ID | | DHR70 | | SKKGSPETAREAVRLALELVQEAARVARKTGSTELLIAAAKLAIEVARVALKVGS |
| NO: 4 | | | | PEAAREAVRAALELVQELIRAARKTGSKEVLEEAAKLALEVALVAAAVGSSEAAA |
| | | | | KAVATAVEALKEAGASEDEIAEIVARVISEVIRILKENGSEYKVICVSVAKIVAE |
| | | | | IVEALKRSGTSEDEIAEIVARVISEVIRTLKESGSDYLIICVCVAIIVAEIVEAL |
| | | | | KRSGTSEDEIAEIVARVISEVIRTLKESGSSYEVIKECVQIIVLAIILALMKSGT |
| | | | | EVEEILLILLRVKTEVRRTLKESGS(GSLEHHHHHH) |
| t32.4 | Protela A | EXT6- | tj04c3_int5v2 | (M)FNKSQQSAFYLILNMPNLNEAQRNGFIQSLKDDPSKSEVVAGEAAIEAARNA |
| (aka | | DHR70 | | LKKGSPETAREAVRLALELVQEAERQARKTGSTERLIAAAKLAIEVARVALKVGS |
| t.4_r1) | | | | PETAREAVRTALELVQELIRQARKTGSKEVLEEAAKLALEVAKVAAEVGSP |
| SEQ ID | | | | ETAARAVATAVEALKEAGASEDEIAEIVARVISEVIRILKESGSEYKVICRAVAR |
| NO: 5 | | | | IVAEIVEALKRSGTSEDEIAEIVARVISEVIRTLKESGSDYLIICVCVAIIVAEI |
| | | | | VEALKRSGTSEDEIAEIVARVISEVIRTLKESGSSYEVIKECVQIIVLAIILALM |
| | | | | KSGTEVEEILLILLRVKTEVRRTLKES(GSLEHHHHHH) |
| t32.8 | Protela A | EXT6- | tj04C3_int5v2 | (M)FNKDQQSAFYEVLNMPNLNEAQRNGFIQSLKDDPSQSLKILIKAAAGGDSEL |
| SEQ ID | | DHR39 | | EEVAKRIVKELAEQGRSEKEAAKEAAELIERITRAAGGNSDLIELAVRIVKILEE |
| NO: 6 | | | | QGRSPSEAAKEAVEAIEAIVRAAGGDSEAIKVAAEIAKTIITQKESGSEYKEICR |
| | | | | TVARIVAEIVEKLKRNGASEDEIAEIVAAIIAAVILTLKLSGSDYLIICVCVAII |
| | | | | VAEIVEALKRSGTSEDEIAEIVARVISAVIRVLKESGSSYEVIKECVQIIVLAII |
| | | | | LALMKSGTEVEEILLILLRVKTEVRRTLKES(GSLEHHHHHH) |
| o42.1 | Protela A | EXT6- | tj10C4_G1 | (M)FNKDQQSAFYEILNMPNLNEALRNGFIQLLKDDPSKSTVILTAAKVAAELSE |
| SEQ ID | | DHR9 | | KIRTLKESGSSYEQIAETVAKAVAKLVEKLKRNGVSEDEIALAVALIISAVIQTL |
| NO: 7 | | | | KESGSSYEVIAEIVARIVAEIVEALKRSGTSEDEIAEIVARVISEVIRTLKESGS |
| | | | | SYEVIAEIVARIVAEIVEALKRSGTSEDEIAKIVARVIAEVLRTLKESGSSEEVI |
| | | | | KEIVARIITEIKEALKRSGTSEDEIELITLMIEAALEIAKLKSSGSEYEEICEDV |
| | | | | ARRIAELVEKLKRDGTSAVEIAKIVAAIISAVIAMLKASGSSYEVICECVARIVA |
| | | | | EIVEALKRSGTSAAIIALIVALVISEVIRTLKESGSSFEVILECVIRIVLEIIEA |
| | | | | LKRSGTSEQDVMLIVMAVLLVVLATLQLS(GSLEHHHHHH) |
| i52.3 | DHR79 | EXT6- | 5H2LD- | (M)SDEEERNELIKRIREAAQRAREAAERTGDPRVRELARELARLAQRAFYLVLH |
| SEQ ID | | DHR71 | 10_5 | DPSSSDVNEALKLIVEAIEAAVRALEAAERAGDPELREDAREAVRLAVEAAEEVQ |
| NO: 8 | | | | RNPSSSTANLLLKAIVALAEALAAAANGDKEKFKKAAESALEIAKRVVEVASKEG |
| | | | | DPEAVLEAAKVALRVAELAAKNGDKEVFKKAAESALEVAKRLVEVASKEGDPELV |
| | | | | LEAAKVALRVAELAAKNGDKEVFQKAAASAVEVALRLTEVASKEGDSELETEAAK |
| | | | | VITRVRELASKQGDAAVAILAETAEVKLEIEESKKRPQSESAKNLILIMQLLINQ |
| | | | | IRLLVLQIRMLDEQRQE(GSLEHHHHHH) |
| i52.6 | DHR79 | EXT6- | 5H2LD- | (M)SDEEERNELIKRIREAAQRAREAAERTGDPRVRELARELARLAQRAFYLVLH |
| SEQ ID | | DHR57 | 10_5 | DPSSSDVNEALKLIVEAIEAAVRALEAAERTGDPKVREEARELVRRAVEAAEEVQ |
| NO: 9 | | | | RNPSSSEVNEKLKAIVVEIEVKVASLEAKEVTDPDKALKIAKKVIELALEAVKEN |
| | | | | PSTEALRAVLEAVRLASEVAKRVTDPDKALKIAKLVIELALEAVKEDPSTDALRA |
| | | | | VLEAVRLASEVAKRVTDPDKALKIAKLVLELAAEAVKEDPSTDALRAAKEA |
| | | | | ERLATEVAKRVTDPKKAREIEMLVLKLQMEAILAETEEVKKEIEESKKRPQSESA |
| | | | | KNLILIMQLLINQIRLLALQIRMLALQLQE(GSLEHHHHHH) |

As detailed in the examples that follow, the polypeptides of the disclosure comprise 3 domains (as reflected in the columns of Table 1):
(1) An (Fc) binding domain;
(2) A helical polypeptide (monomer) that helps position the Fc-binder domain and oligomer domain at the correct orientation to promote higher order structures (sometimes referred to as cages, or nanoparticles); and
(3) An oligomer domain that can associate via non-covalent interactions to form polymers (including but not limited to homo-polymers), such as dimers, trimers, tetramers, or pentamers (C2, C3, C4, or C5 cyclic symmetry, respectively).

In some embodiments, the oligomer domain can self-associate via non-covalent interactions to form a homo-polymer with an identical polypeptide. In another embodiment, the oligomer domain can associate via non-covalent interactions to form a pseudo-polymer with similar polypeptide that has some amino acid sequence differences, so long as each monomer has the required amino acid sequence identity to the reference polypeptide.

The polypeptides of the disclosure fuse these domains at an orientation that when in oligomeric form and combined with IgG, forms the desired higher order structures as detailed herein.

Each monomer polypeptide has two interfaces: (1) A Fc-binding interface (defined for each polypeptide in Table 3); and (2) An oligomerization domain interface (defined for each polypeptide in Table 2). The polypeptides of the disclosure, when expressed, will form a cyclic oligomer with C2, C3, C4, or C5 symmetry via the oligomerization domain. When combined with antibody, a higher order, cage-like, polyhedral structure spontaneously assembles via interaction of the antibodies with Fc binding interfaces. The resulting higher order structures have cyclic symmetry at each Fc-binding interface and each homo-oligomerization domain interface.

As used herein, antibody includes the full length antibodies (heavy and light chain) and any functional antibody fragments that include the IgG fragment crystallizable (Fc) domain. In some embodiments, the antibody includes heavy and light chains. In other embodiments, the antibody may comprise a fusion protein comprising a protein that binds a target and an Fc domains, that dimerizes since the Fc domains naturally dimerizes. In other embodiments, the antibody may comprise an Fc fragment chemically modified to a protein that binds a target, which dimerizes since the Fc domains naturally dimerizes.

In one embodiment, amino acid residues that would be present at a polymeric interface (as defined in Table 2) in a polymer of the polypeptide of any one of SEQ ID NOS: 1-9 are conserved (i.e.: identical to the amino acid residue at the same position in the reference polypeptide).

TABLE 2
Predicted interface residues at oligomeric interface (i.e.,
not the Fc/Fc-binder interface) by residue position

| Name | Interface residue positions |
|---|---|
| d2.3 | 122, 185, 188, 189, 192, 195, 196, 199, 200, 202, 203, 204, 234, 235, 238, |
| SEQ ID | 239, 242, 245, 246, 249, 250, 252, 253, 280, 281, 282, 284, 285, 288, 292, |
| NO: 1 | 295, 296, 299, 303, 338 |
| d2.4 | 185, 188, 189, 192, 195, 196, 199, 200, 202, 203, 235, 238, 239, 242, 245, |
| SEQ ID | 246, 249, 250, 252, 253, 280, 281, 282, 284, 285, 288, 292, 295, 296, 299, |
| NO: 2 | 303, 338 |
| d2.7 | 202, 205, 206, 209, 213, 216, 217, 219, 220, 221, 251, 252, 255, 256, 259, |
| SEQ ID | 262, 263, 266, 267, 269, 270, 297, 298, 299, 301, 302, 305, 309, 312, 313, |
| NO: 3 | 316, 320, 355 |
| t32.4_old | 202, 203, 204, 207, 208, 252, 254, 255, 258, 261, 262, 265, 266, 269, 278, |
| SEQ ID | 308, 312, 315, 316, 318, 319, 320, 322, 323, 325, 326, 327, 328, 329, 330, |
| NO: 4 | 331, 332, 333, 335, 336, 337, 339, 340, 343 |
| t32.4 (aka | 202, 203, 204, 207, 208, 252, 254, 255, 258, 261, 262, 265, 266, 269, 278, |
| t.4_r1) | 308, 312, 315, 316, 318, 319, 320, 322, 323, 325, 326, 327, 328, 329, 330, |
| SEQ ID | 331, 332, 333, 335, 336, 337, 339, 340, 343 |
| NO: 5 | |
| t32.8 | 155, 156, 157, 160, 161, 205, 207, 208, 211, 214, 215, 218, 219, 222, 231, |
| SEQ ID | 232, 261, 265, 268, 269, 271, 272, 273, 275, 276, 278, 279, 280, 281, 282, |
| NO: 6 | 283, 284, 285, 286, 288, 289, 290, 292, 293, 296 |
| o42.1 | 288, 289, 290, 294, 338, 339, 340, 341, 343, 344, 348, 364, 368, 369, 372, |
| SEQ ID | 373, 375, 376, 379, 383, 388, 389, 390, 391, 392, 393, 394, 395, 396, 397, |
| NO: 7 | 398, 400, 401, 402, 404, 405, 408, 409, 412 |
| i52.3 | 201, 205, 209, 236, 248, 252, 255, 277, 281, 282, 284, 286, 287, 289, 290, |
| SEQ ID | 293, 294, 297, 300, 301, 304, 305, 307, 308, 309, 310, 311, 312, 313, 314, |
| NO: 8 | 315, 316, 318, 319, 320, 321, 322, 323, 324, 325, 326, 327, 328, 329, 330, |
| | 331, 332, 333, 334, 335, 336, 337, 338, 339, 340, 341, 343 |
| i52.6 | 203, 206, 276, 282, 285, 288, 289, 292, 293, 295, 296, 299, 302, 303, 306, |
| SEQ ID | 309, 310, 313, 314, 316, 317, 318, 319, 320, 321, 322, 323, 324, 325, 326, |
| NO: 9 | 327, 328, 329, 330, 331, 332, 333, 334, 335, 336, 337, 338, 339, 340, 341, |
| | 342, 343, 344, 345, 346, 347, 348, 349, 350, 351, 352 |

In another embodiment, amino acid residues present at an Fc binding interface as defined in Table 3 are conserved.

TABLE 3

| | Fc binder used | |
|---|---|---|
| Name | in design | Interface residue positions |
| d2.3 | DHR79 | 4, 7, 8, 11, 12, 14, 15, 18, 19, 22, 35, 41, |
| SEQ ID | | 42, 44, 45, 46, 48, 49, 50, 52, 53 |
| NO: 1 | | |
| d2.4 | DHR79 | 4, 7, 8, 11, 12, 14, 15, 18, 19, 22, 35, 41, |
| SEQ ID | | 42, 44, 45, 46, 48, 49, 50, 52, 53 |
| NO: 2 | | |
| d2.7 | DHR79 | 4, 7, 8, 11, 12, 14, 15, 18, 19, 22, 35, 41, |
| SEQ ID | | 42, 44, 45, 46, 48, 49, 50, 52, 53 |
| NO: 3 | | |
| t32.4_old | Protein A | 2, 3, 4, 6, 7, 8, 10, 11, 14, 21, 24, 25, |
| SEQ ID | | 28, 32 |
| NO: 4 | | |
| t32.4 (aka | Protein A | 2, 3, 4, 6, 7, 8, 10, 11, 14, 21, 24, 25, |
| t.4_r1) | | 28, 32 |
| SEQ ID | | |
| NO: 5 | | |
| t32.8 | Protein A | 2, 3, 4, 6, 7, 8, 10, 11, 14, 21, 24, 25, |
| SEQ | | 28, 32 |
| ID NO: 6 | | |
| o42.1 | Protein A | 2, 3, 4, 6, 7, 8, 10, 11, 14, 21, 24, 25, |
| SEQ ID | | 28, 32 |
| NO: 7 | | |
| i52.3 | DHR79 | 4, 7, 8, 11, 12, 14, 15, 18, 19, 22, 35, 41, |
| SEQ ID | | 42, 44, 45, 46, 48, 49, 50, 52, 53 |
| NO: 8 | | |
| i52.6 | DHR79 | 4, 7, 8, 11, 12, 14, 15, 18, 19, 22, 35, 41, |
| SEQ ID | | 42, 44, 45, 46, 48, 49, 50, 52, 53 |
| NO: 9 | | |

In a further embodiment, amino acid substitutions relative to the reference sequence comprise, consist essentially of, or consist of substitutions at polar residues in the reference polypeptide. In other embodiments, polar residues on the surface of the polypeptide that are not at the Fc or oligomeric interfaces may be substituted with other polar residues while maintaining folding and assembly properties of the designs.

As used herein, “polar” residues are C, D, E, H, K, N, Q, R, S, T, and Y. “Non-polar” residues are defined as A, G, I, L, M, F, P, W, and V.

In one embodiment, amino acid substitutions relative to the reference sequence comprise, consist essentially of, or consist of substitutions at polar residues at non-Gly/Pro residues in loop positions, as defined in Table 4, in the reference polypeptide.

TABLE 4
Predicted secondary structure for all listed designs, using pyrosetta's
display_secstruct( ) function. L = Loop, H = Helix

| Name | Sequence |
|---|---|
| d2.3 | LLHHHHHHHHHHHHHHHHHHHHHHHHHHLLHHHHHHHHHHHHHHHHHHHHHHHLLLLHHHHHHHHHHHHHHHHHHHH |
| (SEQ ID | HHHHHHHLLHHHHHHHHHHHHHHHHHHHHHHHLLHHHHHHHHHHHHHHHHHHHHHLLHHHHHHHHHHHHHHHHHHLL |
| NO: 1) | HHHHHHHHHHHHHHHHHHHHHHHHHHHLLLHHHHHHHHHHHHHHHHHHHHHLLLLLHHHHHHHHHHHHHHHHHHHHH |
| | LLLHHHHHHHHHHHHHHHHHHHHHHLLLLHHHHHHHHHHHHHHHHHHHHHLLLHHHHHHHHHHHHHHHHHHHHHHLL |
| | LLHHHHHHHHHHHHHHHHHHHHHLLLHHHHHHHHHHHHHHHHHHHLLLLLLLLLLLL |
| d2.4 | LLHHHHHHHHHHHHHHHHHHHHHHHHHHLLHHHHHHHHHHHHHHHHHHHHHHHLLLLHHHHHHHHHHHHHHHHHHHH |
| (SEQ ID | HHHHHHHLLHHHHHHHHHHHHHHHHHHHHHHHLLHHHHHHHHHHHHHHHHHHHHHLLHHHHHHHHHHHHHHHHHHLL |
| NO: 2) | HHHHHHHHHHHHHHHHHHHHHHHHHHHLLLHHHHHHHHHHHHHHHHHHHHHLLLLLHHHHHHHHHHHHHHHHHHHHH |
| | LLLHHHHHHHHHHHHHHHHHHHHHHLLLLHHHHHHHHHHHHHHHHHHHHHLLLHHHHHHHHHHHHHHHHHHHHHHLL |
| | LLHHHHHHHHHHHHHHHHHHHHHLLLHHHHHHHHHHHHHHHHHHHLLLLLLLLLLLL |
| d2.7 | LLHHHHHHHHHHHHHHHHHHHHHHHHHHLLHHHHHHHHHHHHHHHHHHHHHHHLLLLHHHHHHHHHHHHHHHHHHHH |
| (SEQ ID | HHHHHHHLLHHHHHHHHHHHHHHHHHHHHHHHHHHHHHHHLLHHHHHHHHHHHHHHHHHHHHHHHHHHLLLHHHHHH |
| NO: 3) | HHHHHHHHHHHHHHHHHHLLHHHHHHHHHHHHHHHHHHHHHHHHLLLHHHHHHHHHHHHHHHHHHHHHLLLLLHHHH |
| | HHHHHHHHHHHHHHHHHLLLHHHHHHHHHHHHHHHHHHHHHHLLLLHHHHHHHHHHHHHHHHHHHHHLLLHHHHHHH |
| | HHHHHHHHHHHHHHHLLLLHHHHHHHHHHHHHHHHHHHHHLLLHHHHHHHHHHHHHHHHHHHLLLLLLLLLLLL |
| t32.4_old | LLLHHHHHHHHHHHHLLLLLHHHHHHHHHHHHHLHHHHHHHHHHHHHHHHHHHHHHLLHHHHHHHHHHHHHHHHHHH |
| (SEQ ID | HHHHHHLLHHHHHHHHHHHHHHHHHHHHHLLHHHHHHHHHHHHHHHHHHHHHHHHHLLHHHHHHHHHHHHHHHHHHH |
| NO: 4) | HHLLHHHHHHHHHHHHHHHHHLLLLHHHHHHHHHHHHHHHHHHHHHLLLLHHHHHHHHHHHHHHHHHHHHHLLLLHH |
| | HHLLHHHHHHHHHHHHHHHLLLLHHHHHHHHHHHHHHHHHHHHHHLLLHHHHHHHHHHHHHHHHHHHHHLLLLHHHH |
| | HHHHHHHHHHHHHHHHHLLLLHHHHHHHHHHHHHHHHHHHHHHLLLLLLLLLLLLL |
| t32.4 | LLLHHHHHHHHHHHHLLLLLHHHHHHHHHHHHHLHHHHHHHHHHHHHHHHHHHHHHLLHHHHHHHHHHHHHHHHHHH |
| (aka | HHHHHHLLHHHHHHHHHHHHHHHHHHHHHLLHHHHHHHHHHHHHHHHHHHHHHHHHLLHHHHHHHHHHHHHHHHHHH |
| t.4_r1) | HHLLHHHHHHHHHHHHHHHHHLLLLHHHHHHHHHHHHHHHHHHHHHLLLLHHHHHHHHHHHHHHHHHHHHHLLLLHH |
| (SEQ ID | HHLLHHHHHHHHHHHHHHHLLLLHHHHHHHHHHHHHHHHHHHHHHLLLHHHHHHHHHHHHHHHHHHHHHLLLLHHHH |
| NO: 5) | HHHHHHHHHHHHHHHHHLLLLHHHHHHHHHHHHHHHHHHHHHHLLLLLLLLLLLLL |
| t32.8 | LLLHHHHHHHHHHHHLLLLLHHHHHHHHHHHHHLHHHHHHHHHHHHHLLLHHHHHHHHHHHHHHHHHLLLHHHHHHH |
| (SEQ ID | HHHHHHHHHHHHLLLHHHHHHHHHHHHHHHHHLLLHHHHHHHHHHHHHHHHHHHLLLHHHHHHHHHHHHHHHHHHHL |
| NO: 6) | LLLHHHHHHHHHHHHHHHHHHHHHLLLLHHHHHHHHHHHHHHHHHHHHHLLLLHHHHHHHHHHHHHHHHHHHHHHLL |
| | LHHHHHHHHHHHHHHHHHHHHHLLLLHHHHHHHHHHHHHHHHHHHHHLLLLHHHHHHHHHHHHHHHHHHHHHHLLLL |
| | LLLLLLLLL |
| o42.1 | LLLHHHHHHHHHHHHLLLLLHHHHHHHHHHHHHLHHHHHHHHHHHHHHHHHHHHHHHHHHHLLLHHHHHHHHHHHHH |
| (SEQ ID | HHHHHHHHLLLLHHHHHHHHHHHHHHHHHHHHHHLLLHHHHHHHHHHHHHHHHHHHHHLLLLHHHHHHHHHHHHHHH |
| NO: 7) | HHHHHHHLLLHHHHHHHHHHHHHHHHHHHHHLLLLHHHHHHHHHHHHHHHHHHHHHHLLLHHHHHHHHHHHHHHHHH |
| | HHHHLLLLHHHHHHHHHHHHHHHHHHHHHHHLLLHHHHHHHHHHHHHHHHHHHHHHLLLHHHHHHHHHHHHHHHHHH |
| | HHHHLLLHHHHHHHHHHHHHHHHHHHHHHLLLHHHHHHHHHHHHHHHHHHHHHHLLLHHHHHHHHHHHHHHHHHHHH |
| | HLLLLHHHHHHHHHHHHHHHHHHHHHHLLLLLLLLLLLLL |
| i52.3 | LLHHHHHHHHHHHHHHHHHHHHHHHHHHLLHHHHHHHHHHHHHHHHHHHHHHHLLLLHHHHHHHHHHHHHHHHHHHH |
| (SEQ ID | HHHHHHHLLHHHHHHHHHHHHHHHHHHHHHHHLLLLHHHHHHHHHHHHHHHHHHHHHHLLHHHHHHHHHHHHHHHHH |
| NO: 8) | HHHHHHHHLLHHHHHHHHHHHHHHHHHHHHHLLHHHHHHHHHHHHHHHHHHHHHHHHHLLHHHHHHHHHHHHHHHHH |
| | HHHHLLHHHHHHHHHHHHHHHHHHHHHHHHHLLHHHHHHHHHHHHHHHHHHHHHLLHHHHHHHHHHHHHHHHHHHHH |
| | LLLLHHHHHHHHHHHHHHHHHHHHHHHHHHHHHHHHLLLLLLLLLLLL |
| i52.6 | LLLHHHHHHHHHHHHLLLLLHHHHHHHHHHHHHLHHHHHHHHHHHHHHHHHHHHHHLLHHHHHHHHHHHHHHHHHHH |
| (SEQ ID | HHHHHHLLHHHHHHHHHHHHHHHHHHHHHLLHHHHHHHHHHHHHHHHHHHHHHHHHLLHHHHHHHHHHHHHHHHHHH |
| NO: 9) | HHLLHHHHHHHHHHHHHHHHHLLLLHHHHHHHHHHHHHHHHHHHHHLLLLHHHHHHHHHHHHHHHHHHHHHLLLLHH |
| | HHHHHHHHHHHHHHHHHHHLLLLHHHHHHHHHHHHHHHHHHHHHHLLLHHHHHHHHHHHHHHHHHHHHHLLLLHHHH |
| | HHHHHHHHHHHHHHHHHLLLLHHHHHHHHHHHHHHHHHHHHHHLLLLLLLLLLLLL |

In a further embodiment of any of these embodiments, amino acid changes from the reference polypeptide are conservative amino acid substitutions. As used here, “conservative amino acid substitution” means that:
hydrophobic amino acids (Ala, Cys, Gly, Pro, Met, Sce, Sme, Val, Ile, Leu) can only be substituted with other hydrophobic amino acids;
hydrophobic amino acids with bulky side chains (Phe, Tyr, Trp) can only be substituted with other hydrophobic amino acids with bulky side chains;
amino acids with positively charged side chains (Arg, His, Lys) can only be substituted with other amino acids with positively charged side chains;
amino acids with negatively charged side chains (Asp, Glu) can only be substituted with other amino acids with negatively charged side chains; and
amino acids with polar uncharged side chains (Ser, Thr, Asn, Gln) can only be substituted with other amino acids with polar uncharged side chains.

In one embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 2-3, 5-6, and 8-9.

In all embodiments disclosed herein, the polypeptides may comprise one or more additional functional groups or residues as deemed appropriate for an intended use. The polypeptides of the disclosure may include additional residues at the N-terminus or C-terminus, or a combination thereof; these additional residues are not included in determining the percent identity of the polypeptides of the invention relative to the reference polypeptide. Such residues may be any residues suitable for an intended use, including but not limited to detectable proteins or fragments thereof (also referred to as “tags”). As used herein, “tags” include general detectable moieties (i.e.: fluorescent proteins, antibody epitope tags, etc.), therapeutic agents, purification tags (His tags, etc.), linkers, ligands suitable for purposes of purification, ligands to drive localization of the polypeptide, peptide domains that add functionality to the polypeptides. In non-limiting embodiments, such functional groups may comprise one or more polypeptide antigens, polypeptide therapeutics, enzymes, detectable domains (ex: fluorescent proteins or fragments thereof), DNA binding proteins, transcription factors, etc. In one embodiment, the polypeptides may further comprise a functional polypeptide covalently linked to the amino-terminus and/or the carboxy-terminus. In other embodiments, the functional polypeptide may include, but is not limited to, a detectable polypeptide such as a fluorescent or luminescent polypeptide, receptor binding domains, etc.

The polypeptides described herein may be chemically synthesized or recombinantly expressed. The polypeptides may be linked to other compounds to promote an increased half-life in vivo, such as by PEGylation, HESylation, PASylation, or glycosylation. Such linkage can be covalent or non-covalent as is understood by those of skill in the art.

In another aspect the disclosure provides nucleic acids encoding the polypeptide of any embodiment or combination of embodiments of the disclosure. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of affecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the polypeptides, nucleic acids and/or expression vectors (i.e.: episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection.

The disclosure also provides polypeptide polymers, wherein:
(i) each monomer in the polymer comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:1;
(ii) each monomer in the polymer comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:2;
(iii) each monomer in the polymer comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:3;
(iv) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:4;
(v) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:5;
(vi) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:6;
(vii) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:7;
(viii) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:8; or
(ix) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:9;
wherein optional residues are not considered in the percent identity requirement.

As described herein, the polypeptides of the disclosure, when expressed, will form a cyclic oligomer with C2, C3, C4, or C5 symmetry via the oligomerization domain, generating the polymers of the disclosure.

The polymer may comprise monomers with some amino acid differences, or all monomers in a given polymer may be identical. The polymer may be a dimer, trimer, tetramer, or pentamer.

In one embodiment, the polymer comprises a dimer. In various such embodiments, the dimer comprises a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-3.

In another embodiment, the polymer comprises a trimer. In various such embodiments, the trimer comprises a polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 4-6.

In a further embodiment, the polymer comprises a tetramer. In various such embodiments, the tetramer comprises polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:7.

In a still further embodiment, the polymer comprises a pentamer. In various such embodiments, the pentamer comprises a polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:8-9.

In another aspect, the disclosure provides particles, comprising:
(a) a plurality of identical polymers according to any embodiment dor combination of embodiments disclosed herein, and
(b) a plurality of antibodies comprising Fc domains;
wherein:
   (i) each antibody in the plurality of antibodies comprises a first Fc domain and a second Fc domain;
   (ii) each antibody in the plurality of antibodies is (A) non-covalently bound via the first Fc domain to one polypeptide monomer chain of a first homo-polymer, and (B) non-covalently bound via the second Fc domain to one polypeptide monomer of a second homo-polymer; and
   (iii) each polypeptide monomer chain of each homo-polymer is non-covalently bound to one Fc domain;
wherein the particle comprises dihedral, tetrahedral, octahedral, or icosahedral symmetry.

As described herein, the polypeptides of the disclosure, when expressed, will form a cyclic oligomer with C2, C3, C4, or C5 symmetry via the oligomerization domain. When combined with antibody, a higher order, cage-like, polyhedral structure spontaneously assembles via interaction of the antibodies with Fc binding interfaces. The resulting higher order structures have C2 cyclic symmetry at the Fc position and cyclic 2, 3, 4, or 5-symmetry at each oligomerization domain interface. The resulting particles form precisely ordered and structurally homogeneous antibody-bound nanoparticle structures. As such, the particles can be used, for example, in any therapeutic or diagnostic use for which the antibodies provide a benefit.

As used herein, antibody includes full length antibodies (heavy and light chain) and any functional antibody fragments that include the IgG fragment crystallizable (Fc) domain. In some embodiments, the antibody includes heavy and light chains. In other embodiments, the antibody may comprise a fusion protein comprising a protein that binds a target and an Fc domain, that dimerizes since the Fc domains naturally dimerizes. In other embodiments, the antibody may comprise an Fc fragment chemically modified to a protein that binds a target, which dimerizes since the Fc domains naturally dimerizes. The polypeptides of the disclosure bind to the antibody constant region, and thus the antibody can be an antibody with specificity for any antigen.

In one embodiment, the plurality of homo-polymers comprises homo-dimers of the polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-3. In these embodiments, adding the recited polypeptides with IgG results in spontaneous assembly into a D2 dihedral structure containing two antibodies per particle.

In another embodiment, the plurality of homo-polymers comprises homo-trimers of the polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 4-6. In these embodiments, adding the recited polypeptides with IgG results in spontaneous assembly into a T32 tetrahedral structure containing six antibodies per particle.

In a further embodiment, the plurality of homo-polymers comprises homo-tetramers of the polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:7. In these embodiments, adding the recited polypeptides with IgG results in spontaneous assembly into an O42 octahedral structure containing twelve antibodies per particle.

In a still further embodiment, the plurality of homo-polymers comprises homo-pentamers of the polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 8-9. In these embodiments, adding the recited polypeptides with IgG results in spontaneous assembly into an I52 icosahedral structure containing thirty antibodies per particle.

In another embodiment, the particles comprise:
(a) a plurality of polypeptide polymers, wherein
   (i) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:1;
   (ii) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:2;
   (iii) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:3;
   (iv) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:4;
   (v) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:5;
   (vi) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:6;
   (vii) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:7;
   (viii) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:8; or
   (ix) each monomer in the polymers comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:9; wherein residues in parentheses are optional (i.e.: not considered in the percent identity requirement); and
(b) a plurality of antibodies comprising Fc domains, wherein
   (i) each antibody in the plurality of antibodies comprises a first Fc domain and a second Fc domain;
   (ii) each antibody in the plurality of antibodies is (A) non-covalently bound via the first Fc domain to one polypeptide monomer chain of a first polymer, and (B) non-covalently bound via the second Fc domain to one polypeptide monomer of a second polymer; and
   (iii) each polypeptide monomer chain of each polymer is non-covalently bound to one Fc domain;
wherein the particle comprises dihedral, tetrahedral, octahedral, or icosahedral symmetry.

All embodiments described above for the polypeptides and polymers are equally applicable for the particles of the disclosure. In one embodiment, the polymers comprise monomers with some amino acid differences. In another embodiment, the particle comprises polymers that are not homo-oligomers. In a further embodiment, each polymer in the particle is identical.

In another embodiment, each monomer in each polymer is identical and each polymer is a homo-polymer. In a further embodiment, each homo-polymer in the particle is identical.

In one embodiment, the plurality of polymers comprises dimers of the polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-3. In these embodiments, adding the recited polypeptides with antibodies results in spontaneous assembly into a D2 dihedral structure containing two antibodies per particle.

In another embodiment, the plurality of polymers comprises trimers of the polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 4-6. In these embodiments, adding the recited polypeptides with antibodies results in spontaneous assembly into a T32 tetrahedral structure containing six antibodies per particle.

In a further embodiment, the plurality of polymers comprises tetramers of the polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:7. In these embodiments, adding the recited polypeptides with antibodies results in spontaneous assembly into an O42 octahedral structure containing twelve antibodies per particle.

In a still further embodiment, the plurality of polymers comprises pentamers of the polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 8-9. In these embodiments, adding the recited polypeptides with antibodies results in spontaneous assembly into an I52 icosahedral structure containing thirty antibodies per particle.

In one embodiment of all embodiments of the particles, amino acid residues present at a polymeric interface, as defined in Table 2, in a polymer of the polypeptide of any one of SEQ ID NOS: 1-9 are conserved. In another embodiment of all embodiments of the particles, amino acid residues present at a Fc binding interface of any one of SEQ ID NOS: 1-9 as defined in Table 3 are conserved. In a further embodiment of all embodiments of the particles, amino acid substitutions relative to the reference sequence of any one of SEQ ID NOS: 1-9 comprise, consist essentially of, or consist of substitutions at polar residues in the reference polypeptide. In a still further embodiment of all embodiments of the particles amino acid substitutions relative to the reference sequence of any one of SEQ ID NOS: 1-9 comprise, consist essentially of, or consist of substitutions at polar residues at non-Gly/Pro residues in loop positions, as defined in Table 4, in the reference polypeptide. In another embodiment of all embodiments of the particles, amino acid changes from the reference polypeptide of any one of SEQ ID NOS: 1-9 are conservative amino acid substitutions.

The antibodies present in the particles may be any suitable antibody for an intended purpose. In one embodiment, the antibodies selectively bind to a target including, but not limited to, a pathogen-specific antigen (including but not limited to bacterial, viral, protozoan, or other pathogen antigen), a cell surface receptor, a disease-related antigen (including but not limited to a tumor cell antigen, beta amyloid for Alzheimer's and other amyloid-based diseases), enzymes, growth factors, toxins, small molecules, peptides of diagnostic interest, etc.

In another embodiment, the antibodies may comprise one or more of the FDA-approved antibodies for therapeutic uses as noted in Table 5. In these embodiments, the particles can be used, for example, to treat the disorder(s) for which the antibodies are approved against, as noted in the right hand column of Table 5.

TABLE 5

| | | Indication |
|---|---|---|
| Antibody | Target | (Targeted disease) |
| abciximab | GPIIb/IIIa | Percutaneous coronary intervention |
| adalimumab | TNF | Rheumatoid arthritis |
| adalimumab-adbm | TNF | Rheumatoid arthritis |
| | | Juvenile idiopathic arthritis |
| | | Psoriatic arthritis |
| | | Ankylosing spondylitis |
| | | Crohn's disease |
| | | Ulcerative colitis |
| | | Plaque psoriasis |
| adalimumab-atto | TNF | Rheumatoid arthritis |
| | | Juvenile idiopathic arthritis |
| | | Psoriatic arthritis |
| | | Ankylosing spondylitis |
| | | Crohn's disease |
| | | Ulcerative colitis |
| | | Plaque psoriasis |
| ado-trastuzumab | HER2 | Metastatic breast cancer |
| emtansine | | |
| alemtuzumab | CD52 | B-cell chronic lymphocytic leukemia |
| alirocumab | PCSK9 | Heterozygous familial hypercholesterolemia |
| | | Refractory hypercholesterolemia |
| atezolizumab | PD-L1 | Urothelial carcinoma |
| atezolizumab | PD-L1 | Urothelial carcinoma |
| | | Metastatic non-small cell lung cancer |
| avelumab | PD-L1 | Metastatic Merkel cell carcinoma |
| basiliximab | IL2RA | Prophylaxis of acute organ rejection in renal |
| | | transplant |
| belimumab | BLyS | Systemic lupus erythematosus |
| benralizumab | interleukin-5 receptor | Severe asthma, eosinophilic phenotype |
| | alpha subunit | |
| bevacizumab | VEGF | Metastatic colorectal cancer |
| bevacizumab-awwb | VEGF | Metastatic colorectal cancer |
| | | Non-squamous Non-small-cell lung carcinoma |
| | | Glioblastoma |
| | | Metastatic renal cell carcinoma |
| | | Cervical cancer |
| bezlotoxumab | Clostridium difficile | Prevent recurrence of Clostridium difficile infection |
| | toxin B | |
| blinatumomab | CD19 | Precursor B-cell acute lymphoblastic leukemia |
| brentuximab vedotin | CD30 | Hodgkin lymphoma |
| | | Anaplastic large-cell lymphoma |
| brodalumab | IL17RA | Plaque psoriasis |
| burosumab-twza | FGF23 | X-linked hypophosphatemia |
| canakinumab | IL1B | Cryopyrin-associated periodic syndrome |
| capromab pendetide | PSMA | Diagnostic imaging agent in newly |
| | | diagnosed prostate cancer or post-prostatectomy |
| certolizumab pegol | TNF | Crohn's disease |
| cetuximab | EGFR | Metastatic colorectal carcinoma |
| daclizumab | IL2RA | Prophylaxis of acute organ rejection in renal |
| | | transplant |
| daclizumab | IL2R | Multiple sclerosis |
| daratumumab | CD38 | Multiple myeloma |
| denosumab | RANKL | Postmenopausal women with osteoporosis |
| dinutuximab | GD2 | Pediatric high-risk neuroblastoma |
| dupilumab | IL4RA | Atopic dermatitis, asthma |
| durvalumab | PD-L1 | Urothelial carcinoma |
| eculizumab | Complement | Paroxysmal nocturnal hemoglobinuria |
| | component 5 | |
| elotuzumab | SLAMF7 | Multiple myeloma |
| emicizumab-kxwh | Factor IXa, Factor X | Hemophilia A (congenital Factor VIII deficiency) |
| | | with Factor VIII inhibitors. |
| erenumab-aooe | CGRP receptor | Migraine headache prevention |
| evolocumab | PCSK9 | Heterozygous familial hypercholesterolemia |
| | | Refractory hypercholesterolemia |
| gemtuzumab ozogamicin | CD33 | Acute myeloid leukemia |
| golimumab | TNF | Rheumatoid arthritis |
| | | Psoriatic arthritis |
| | | Ankylosing spondylitis |
| golimumab | TNF | Rheumatoid arthritis |
| guselkumab | IL23 | Plaque psoriasis |
| ibalizumab-uiyk | CD4 | HIV |
| ibritumomab | CD20 | Relapsed or refractory low-grade, follicular, or |
| tiuxetan | | transformed B-cell non-Hodgkin's lymphoma |
| idarucizumab | dabigatran | Emergency reversal of anticoagulant dabigatran |
| infliximab | TNF alpha | Crohn's disease |
| infliximab-abda, | TNF | Crohn's disease |
| infliximab-dyyb, | | Ulcerative colitis |
| infliximab-qbtx | | Rheumatoid arthritis |
| | | Ankylosing spondylitis |
| | | Psoriatic arthritis |
| | | Plaque psoriasis |
| inotuzumab ozogamicin | CD22 | Precursor B-cell acute lymphoblastic leukemia |
| ipilimumab | CTLA-4 | Metastatic melanoma |
| ixekizumab | IL17A | Plaque psoriasis |
| mepolizumab | IL5 | Severe asthma |
| natalizumab | alpha-4 integrin | Multiple sclerosis |
| necitumumab | EGFR | Metastatic squamous non-small cell lung carcinoma |
| nivolumab | PD-1 | Metastatic melanoma |
| nivolumab | PD-1 | Metastatic squamous non-small cell lung carcinoma |
| obiltoxaximab | Protective antigen of | Inhalational anthrax |
| | the Anthrax toxin | |
| obinutuzumab | CD20 | Chronic lymphocytic leukemia |
| ocrelizumab | CD20 | Multiple sclerosis |
| ofatumumab | CD20 | Chronic lymphocytic leukemia |
| olaratumab | PDGFRA | Soft tissue sarcoma |
| omalizumab | IgE | Moderate to severe persistent asthma |
| palivizumab | F protein of RSV | Respiratory syncytial virus |
| panitumumab | EGFR | Metastatic colorectal cancer |
| pembrolizumab | PD-1 | Metastatic melanoma |
| pertuzumab | HER2 | Metastatic breast cancer |
| ramucirumab | VEGFR2 | Gastric cancer |
| ranibizumab | VEGFR1 | Wet age-related macular degeneration |
| | VEGFR2 | |
| raxibacumab | Protective antigen of | Inhalational anthrax |
| | Bacillus anthracis | |
| reslizumab | IL5 | Severe asthma |
| rituximab | CD20 | B-cell non-Hodgkin's lymphoma |
| rituximab and | CD20 | Follicular lymphoma |
| hyaluronidase | | Diffuse large B-cell lymphoma |
| | | Chronic lymphocytic leukemia |
| sarilumab | IL6R | Rheumatoid arthritis |
| secukinumab | IL17A | Plaque psoriasis |
| siltuximab | IL6 | Multicentric Castleman's disease |
| tildrakizumab-asmn | IL23 | Plaque psoriasis |
| tocilizumab | IL6R | Rheumatoid arthritis |
| tocilizumab | IL6R | Rheumatoid arthritis |
| | | Polyarticular juvenile idiopathic arthritis |
| | | Systemic juvenile idiopathic arthritis |
| trastuzumab | HER2 | Metastatic breast cancer |
| trastuzumab-dkst | HER2 | HER2-overexpressing breast cancer, metaststic |
| | | gastric or gastroesophageal junction adenocarcinoma |
| ustekinumab | IL12 | Plaque psoriasis |
| | IL23 | Psoriatic arthritis |
| | | Crohn's disease |
| vedolizumab | integrin receptor | Ulcerative colitis |
| | | Crohn's disease |

In another embodiment, the antibody may selectively bind an antigen from a bacterial or viral pathogen. In non-limiting embodiments, the pathogen-specific antigens include antigens from hepatitis (A, B, C, E, etc.) virus, human papillomavirus, herpes simplex viruses, cytomegalovirus, coronaviruses including but not limited to MERS-CoV (Middle East respiratory syndrome-related coronavirus), and Severe acute respiratory syndrome-related coronavirus (including SARS-CoV and SARS-CoV-2), Epstein-Barr virus, influenza virus, parainfluenza virus, enterovirus, measles virus, mumps virus, polio virus, rabies virus, human immunodeficiency virus, respiratory syncytial virus, Rotavirus, rubella virus, varicella zoster virus, Ebola virus, cytomegalovirus, Marburg virus, norovirus, variola virus, any Flavivus including but not limited to West Nile virus, yellow fever virus, dengue virus, tick-borne encephalitis virus, and Japanese encephalitis virus; human immunodeficiency virus (HIV), Bacillus anthracis, Bordetalla pertusis, Chlamydia trachomatis, Clostridium tetani, Clastridium difficile, Corynebacterium diptheriae, Coxiella burnetii, Escherichia coli, Haemophilus influenza, Helicobacter pylori, Leishmania donovani, L. tropica and L. braziliensis, Mycobacterium tuberculosis, Mycobacterium leprae, Neisseria meningitis, Plasmodium falciparum, P. ovale, P. malariae and P. vivax, Pseudomonas aeruginosa, Salmonella typhi, Schistosoma hematobium, S. mansoni, Streptococcus pneumoniae (group A and B), Staphylococcus aureus, Toxoplasma gondii, Trypanosoma brucei, T. cruzi and Vibrio cholera. In these embodiments, the particles can be used, for example, to treat or limit development of a bacterial or viral infection.

As described in the examples, the as particles have substantial internal volume that can be used to package nucleic acid or protein cargo. Thus, in another embodiment that can be combined with any other embodiment, the particles comprise a cargo within the particle internal volume. Any suitable cargo may be packaged within the particles, including but not limited to nucleic acids or polypeptides useful for an intended purpose.

In another embodiment, the disclosure provides compositions, comprising a plurality of the particles of any embodiment or combination of embodiments herein. The compositions can be used, for example, for therapeutics or diagnostic purposes as described above. In one embodiment, all antibodies in the composition are selective for the same antigen. In another embodiment, the antibodies in the composition are, in total, selective for two or more (3, 4, 5, 6, 7, 8, 9, 10, or more) different antigens.

In another aspect, the disclosure provides pharmaceutical composition comprising (a) the polypeptides, polymers, particles, or compositions of any embodiment or combination of embodiments herein, and (b) a pharmaceutically acceptable carrier. The pharmaceutical compositions may further comprise (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The composition may also include a lyoprotectant, e.g, sucrose, sorbitol or trehalose. In certain embodiments, the composition includes a preservative e.g, benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80) polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the nanostructure, in lyophilized or liquid form. Exemplary stabilizers include sucrose, sorbitol, glycine, inositol, sodium chloride, methionine, arginine, and arginine hydrochloride.

The polypeptides, polymers, particles, or compositions may be the sole active agent in the composition, or the composition may further comprise one or more other agents suitable for an intended use.

In a further aspect, the disclosure provides methods for use of the polypeptides, nucleic acids, expression vectors, host cells, polymers, particles, compositions, or pharmaceutical compositions for any suitable use, including but not limited to those described in the examples. In one embodiment, the methods comprise administering to a subject (such as a mammal, including but not limited to a human) in need thereof a particle, composition, or pharmaceutical composition of the disclosure, wherein the subject has a disorder that can be treated by the antibody present in the particle, composition, or pharmaceutical composition of the disclosure, and wherein administering of an amount effective of the particle, composition, or pharmaceutical composition of the disclosure serves to treat the disorder in the subject. Exemplary such antibodies and disorders that they treat are listed in Table 5. In other embodiments, the disorder is a bacterial or viral infection, and the antibody binds a bacterial or viral antigen. Exemplary such embodiments are provided above.

In another embodiment, the methods comprise administering to a subject (such as a mammal, including but not limited to a human) in need thereof a particle, composition, or pharmaceutical composition of the disclosure, wherein the subject is at risk of developing a disorder whose development can be limited by the antibody present in the particle, composition, or pharmaceutical composition of the disclosure, and wherein administering of an amount effective of the particle, composition, or pharmaceutical composition of the disclosure serves to limit development of the disorder in the subject. In other embodiments, the infection is a bacterial or viral infection, and the antibody binds a bacterial or viral antigen. Exemplary such embodiments are provided above.

As used herein, “treat” or “treating” means accomplishing one or more of the following: (a) reducing severity of symptoms of the disorder in the subject; (b) limiting increase in symptoms in the subject; (c) increasing survival; (d) decreasing the duration of symptoms; (e) limiting or preventing development of symptoms; and (f) decreasing the need for hospitalization and/or the length of hospitalization for treating the disorder.

As used herein, “limiting” means to limit development of the disorder in subjects at risk of such disorder.

As used herein, an “amount effective” refers to an amount of the particle, composition, or pharmaceutical composition that is effective for treating and/or limiting development of the disorder. The particle, composition, or pharmaceutical composition of any embodiment herein are typically formulated as a pharmaceutical composition, such as those disclosed above, and can be administered via any suitable route, including orally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. Polypeptide compositions may also be administered via microspheres, liposomes, immune-stimulating complexes (ISCOMs), or other microparticulate delivery systems or sustained release formulations introduced into suitable tissues (such as blood). Dosage regimens can be adjusted to provide the optimum desired response (e.g., a therapeutic or prophylactic response). A suitable dosage range may, for instance, be 0.1 μg/kg-100 mg/kg body weight of the particle, composition, or pharmaceutical composition thereof. The composition can be delivered in a single bolus, or may be administered more than once (e.g., 2, 3, 4, 5, or more times) as determined by attending medical personnel.

In another aspect, the disclosure provides a polypeptide computational design method as disclosed in any embodiment described in the examples that follow.

### EXAMPLES

We set out to design proteins that drive the assembly of arbitrary antibodies into symmetric assemblies with well-defined structures. We reasoned that symmetric protein assemblies could be built out of IgG antibodies, which are two-fold symmetric proteins, by placing the symmetry axes of the antibodies on the two-fold axes of the target architecture and designing a second protein to hold the antibodies in the correct orientation. As we aimed for a format that would work for many different antibodies, we chose as the nanoparticle interface the interaction between the constant fragment crystallizable (Fc) domain of IgG and the Fc-binding helical bundle protein A.

### A General Computational Method for Antibody Cage Design

To design a homo-oligomer terminating with an Fc-binding interface that has the correct geometry to hold the IgGs in the correct relative orientation for the desired architecture, we computationally fused three protein building blocks together: Fc-binders, monomers, and homo-oligomers. The Fc-binder forms the first nanocage interface between the antibody and the nanocage-forming design, the homo-oligomer forms the second nanocage interface between designed protein chains, and the monomer links the two interfaces together in the correct orientation to generate the desired nanomaterial.

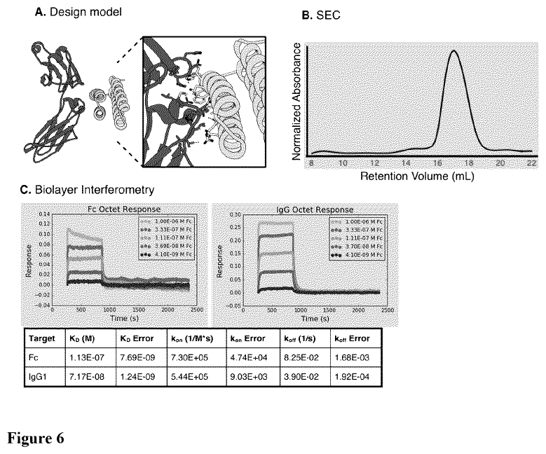

To generate usable Fc-binding building blocks beyond protein A itself, we designed a second Fc-binding building block by grafting the protein A interface residues onto a designed helical repeat protein (FIG. 6). To create designs predicted to form antibody nanocages (hereafter AbCs, for Antibody Cage), we used a library consisting of these 2 Fc-binding proteins, 42 de novo designed helical repeat protein monomers, and between 1-3 homo-oligomers (2 C2s, 3 C3s, 1 C4, and 1 C5). An average of roughly 150 residues were available for fusion per protein building block, avoiding all positions involved in any protein-protein interface, leading to on the order of 107 possible tripartite (Fc-binder/monomer/homo-oligomer) fusions. For each of these tripartite fusions, the rigid body transform between the internal homo-oligomeric interface and the Fc-binding interface is determined by the shapes of each of its three building blocks and the locations and geometry of the “junctions” that link them into a single subunit.

We used a recently described computational protocol (WORMS) that rapidly samples all possible fusions from our building block library to identify those with the net rigid body transforms required to generate dihedral, tetrahedral, octahedral, and icosahedral AbCs (20, 21). To describe the final nanocage architectures, we follow a naming convention which summarizes the point group symmetry and the cyclic symmetries of the building blocks. For example, a T32 assembly has tetrahedral point group symmetry and is built out of a C3 cyclic symmetric antibody-binding designed oligomer, and the C2 cyclic symmetric antibody Fc. While the antibody dimer aligns along the two-fold axis in all architectures, the designed component is a second homodimer in D2 dihedral structures; a homotrimer in T32 tetrahedral structures, O32 octahedral structures, and I32 icosahedral structures; a homotetramer in O42 octahedral structures; and a homopentamer in I52 icosahedral structures.

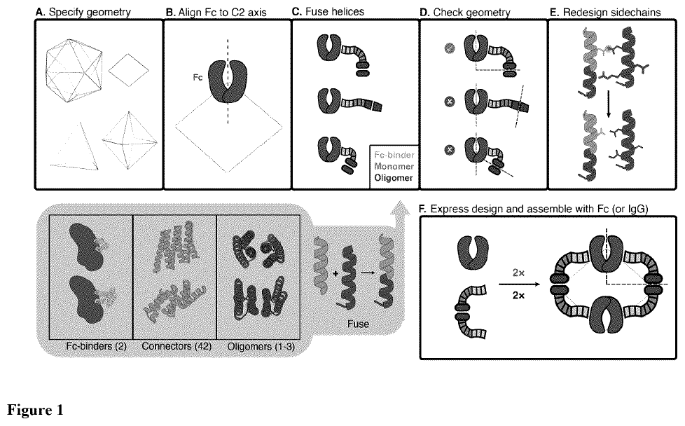

To make the fusions, the protocol first aligns the model of the Fc and Fc-binder protein along the C2 axis of the specified architecture (FIG. 1a-b). The Fc-binder is then fused to a monomer, which is in turn fused to a homo-oligomer. Rigid helical fusions are made by superimposing residues in alpha helical secondary structure from each building block; in the resulting fused structure one building block chain ends and the other begins at the fusion point, forming a new, continuous alpha helix (FIG. 1c). For proper nanocage assembly to occur, fusions are made so that the antibody two-fold axis and the symmetry axis of the homo-oligomer intersect at precise angles at the center of the architecture (FIG. 1d). To generate D2 dihedral, T32 tetrahedral, O32 or O42 octahedral, and I32 or I52 icosahedral nanocages, the required respective intersection angles are 90.0°, 54.7°, 35.3°, 45.0°, 20.9°, and 31.7°. We allowed angular and distance deviations from the ideal architecture of at most 5.7° and 0.5 Å, respectively (see Methods). Candidate fusion models were further filtered based on the number of contacts around the fusion junction (to gauge structural rigidity) and clashes between backbone atoms. Next, the amino acid identities and conformations around the newly formed building block junction were optimized using the SymPackRotamersMover in Rosetta™ to maintain the rigid fusion geometry required for assembly (FIG. 1e). Following sequence design, we selected for experimental characterization six D2 dihedral, eleven T32 tetrahedral, four O32 octahedral, two O42 octahedral, fourteen I32 icosahedral, and eleven I52 icosahedral designs predicted to form AbCs (FIG. 1f).

### Structural Characterization

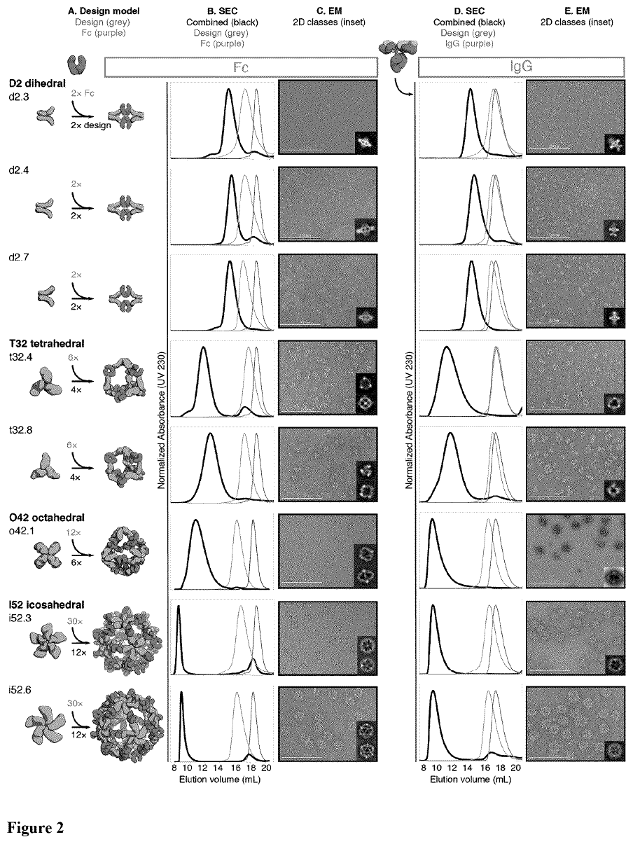

Synthetic genes encoding designed protein sequences appended with a C-terminal 6×histidine tag were expressed in E. coli. Designs were purified from clarified lysates using immobilized metal affinity chromatography (IMAC), and size exclusion chromatography (SEC) was used as a final purification step. Across all geometries, 34 out of 48 AbC-forming designs had a peak on SEC that roughly corresponded to the expected size of the design model. Designs were then combined with human IgG1 Fc, and the assemblies were re-purified via SEC. Eight of these AbC-forming designs assembled with Fc into a species that eluted as a monodisperse peak at a volume consistent with the target nanoparticle molecular weight (FIG. 2a-b; 3 D2 dihedral, 2 T32 tetrahedral, 1 O42 octahedral, and 2 I52 icosahedral AbCs). For the i52.6 design, adding 100 mM L-arginine to the assembly buffer prevented aggregation after combining with Fc; all other designs readily self-assembled in Tris-buffered saline. Most other designs still bound Fc, as evidenced by SEC, native gels, or by visibly precipitating with Fc after combination, but did not form monodisperse nanoparticles by SEC (Table 6), perhaps because of deviations from the target fusion geometry.

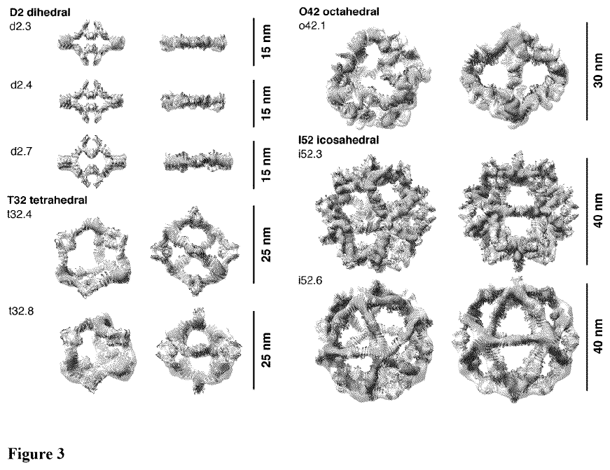

TABLE 6
Success rates of designed antibody-binding cage-forming oligomers.
Solubility (column 2) refers to the presence of protein in
the post-lysis, post-centrifugation, pre-IMAC soluble fraction
as read out by SDS gel. Good SEC component (column 3) refers
to SEC traces with some peak corresponding to the approximate
predicted size of the nanocage-forming design model. Data
for cage formation with Fc are shown in FIG. 2 and 3.

| | | Soluble | Good SEC | Forms cage |
|---|---|---|---|---|
| Geometry | # ordered | component | component | with Fc |
| D2 dihedron | 6 | 5 | 4 | 3 |
| T32 tetrahedron | 11 | 8 | 7 | 2 |
| O32 octahedron | 4 | 3 | 3 | 0 |
| O42 octahedron | 2 | 1 | 1 | 1 |
| I32 icosahedron | 14 | 14 | 10 | 0 |
| I52 icosahedron | 11 | 11 | 10 | 2 |
| Total | 48 | 42 | 35 | 8 |

NS-EM micrographs and two-dimensional class averages revealed nanocages with shapes and sizes corresponding to the design models (FIG. 2c). AbCs also formed when assembled with intact antibodies (IgG with Fc and Fab domains), again generating monodisperse nanocages as shown by SEC and NS-EM (FIG. 2d-e). There is considerably more evidence of flexibility in the electron micrographs of the IgG-AbCs than the Fc-AbCs, as expected given the flexibility of the Fc-Fab hinge. In all cases, 2D class averages collected from the NS-EM data of AbCs made with intact IgG were still able to resolve density corresponding to the non-flexible portion of the assembly (FIG. 2e).

Single-particle NS-EM and cryo-EM reconstructed 3D maps of the AbCs formed with Fc are in close agreement with the computational design models (FIG. 3). Negative-stain EM reconstructions for the dihedral (d2.3, d2.4, d2.7), tetrahedral (t32.4, t32.8), and one of the icosahedral (i52.6) nanocages clearly show dimeric “U”-shaped Fcs and longer designed protein regions that fit together as computationally predicted. A single-particle cryo-EM reconstruction for the o42.1 design has clear density for the six designed tetramers sitting at the C4 vertices, which twist along the edges of the octahedral architecture to bind twelve dimeric Fcs, leaving the eight C3 faces unoccupied. Cryo-EM density for i52.3 with Fc likewise recapitulates the 20-faced shape of a regular icosahedron, with 12 designed pentamers protruding outwards at the C5 vertices (due to the longer length of the C5 building block compared to the monomer or Fc-binder), binding to 30 dimeric Fcs at the center of the edge, with 20 unoccupied C3 faces. In all cases, the computationally designed models fit clearly into the EM densities

### Enhancing Cell Signaling with AbCs

The designed AbCs provide a general platform for investigating the effect of associating cell surface receptors into clusters on signaling pathway activation. Binding of antibodies to cell surface receptors can result in antagonism of signaling as engagement of the natural ligand is blocked (25). While in some cases receptor clustering has been shown to result in activation (11, 26, 27), there have been no systematic approaches to varying the valency and geometry of receptor engagement that can be readily applied to many different signaling pathways. We took advantage of the fact that almost any receptor-binding antibody, of which there are many, can be readily assembled into a wide array of different architectures using our AbC-forming designs to investigate the effect of receptor clustering on signaling. We assembled antibodies and Fc-fusions targeting a variety of signaling pathways into nanoparticles and investigated their effects as described in the following paragraphs.

### Induction of Tumor Cell Apoptosis by α-DR5 Nanocages

Death Receptor 5 (DR5) is a tumor necrosis factor receptor (TNFR) superfamily cell surface protein that initiates a caspase-mediated apoptotic signaling cascade terminating in cell death when cross-linked by its trimeric native ligand. TNF-related apoptosis-inducing ligand (TRAIL) (9, 10, 27-30). Like other members of the family, DR5 can also form alternative signaling complexes that activate non-apoptotic signaling pathways such as the NF-κB pro-inflammatory pathway and pathways promoting proliferation and migration upon ligand binding (29). Because DR5 is overexpressed in some tumors, multiple therapeutic candidates have been developed to activate DR5, such as α-DR5 mAbs and recombinant TRAIL, but these have failed clinical trials due to low efficacy and the development of TRAIL resistance in tumor cell populations (29, 30). Combining trimeric TRAIL with bivalent α-DR5 IgG leads to a much stronger apoptotic response than either component by itself, likely due to induction of larger-scale DR5 clustering via the formation of two-dimensional arrays on the cell surface (27).

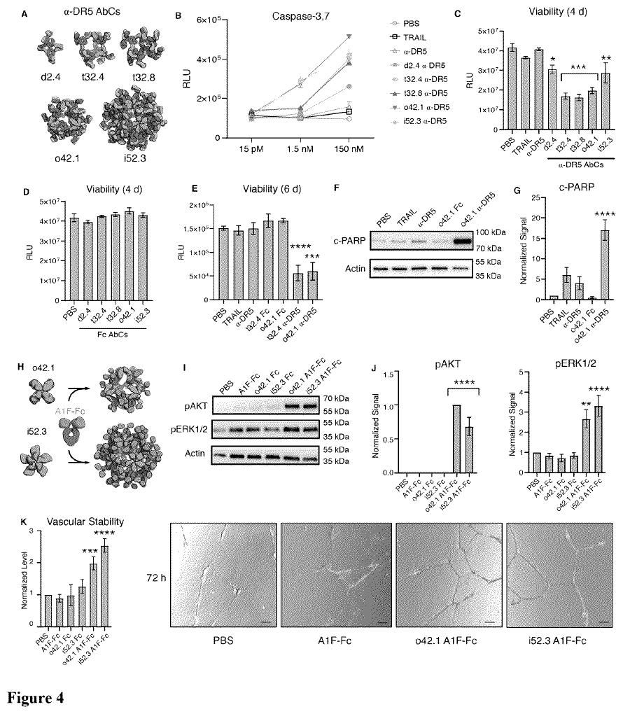

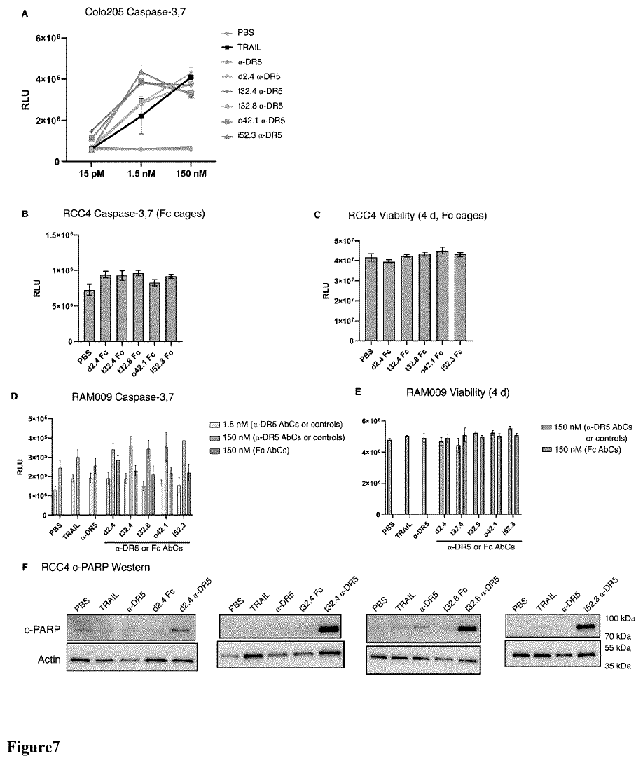

We investigated whether α-DR5 AbCs formed with the same IgG (conatumumab) could have a similar anti-tumor effect without the formation of unbounded arrays. Five designs across four geometries were chosen (d2.4, t32.4, t32.8, o42.1, and i52.3) to represent the range of valencies and shapes (FIG. 4a). All α-DR5 AbCs were found to form single peaks on SEC and yielded corresponding NS-EM micrographs that were consistent with the formation of assembled particles (FIG. 2d-e). All five α-DR5 AbCs caused caspase 3/7-mediated apoptosis at similar levels to TRAIL in a colorectal tumor cell line, whereas the antibody alone or AbCs formed with bare Fc did not lead to caspase-3/7 activity or cell death, even at the highest concentrations tested (FIG. 7a). On the TRAIL-resistant renal cell carcinoma line RCC4, we found that all α-DR5 AbCs induced caspase-3,7 activity (FIG. 4b) and designs t32.4, t32.8, and o42.1 greatly reduced cell viability at 150 nM concentration (FIG. 4c). Free α-DR5 antibody or Fc-only AbCs did not activate caspase, and while TRAIL was found to activate caspase-3,7, this did not lead to a significant decrease in cell viability after four days (FIG. 4b-c, FIG. 7b-c). Designs t32.4 and o42.1 activated caspase at 100-fold lower concentrations (1.5 nM), and prolonged treatment of RCC4 with α-DR5 AbCs t32.4 and o42.1 at 150 nM resulted in the killing of nearly all cells after six days, suggesting that RCC4 cells do not acquire resistance to the nanocages (FIG. 4d). The α-DR5 AbCs did not induce apoptosis in healthy primary kidney tubular cells (FIG. 7d-e).

We next investigated the downstream pathways activated by the α-DR5 AbCs by analyzing their effects on cleaved PARP, a measure of apoptotic activity, as well as the NF-kB target cFLIP. Consistent with the caspase and cell viability data, o42.1 α-DR5 AbCs increased cleaved PARP, while free α-DR5 antibody, TRAIL or o42.1 Fc AbCs did not result in an increase in cleaved PARP over baseline (FIG. 4e-f). We also observed a decrease of cFLIP expression in RCC4 cells after treatment with o42.1 α-DR5 octahedral AbC at 150 nM (FIG. 4e-f). These results suggest that α-DR5 AbCs may overcome TRAIL resistance by inhibiting anti-apoptotic pathways, which enhances the apoptotic cascade induced by DR5 super-clustering.

### Tie-2 Pathway Activation by Fc-Angiopoietin 1 Nanocages

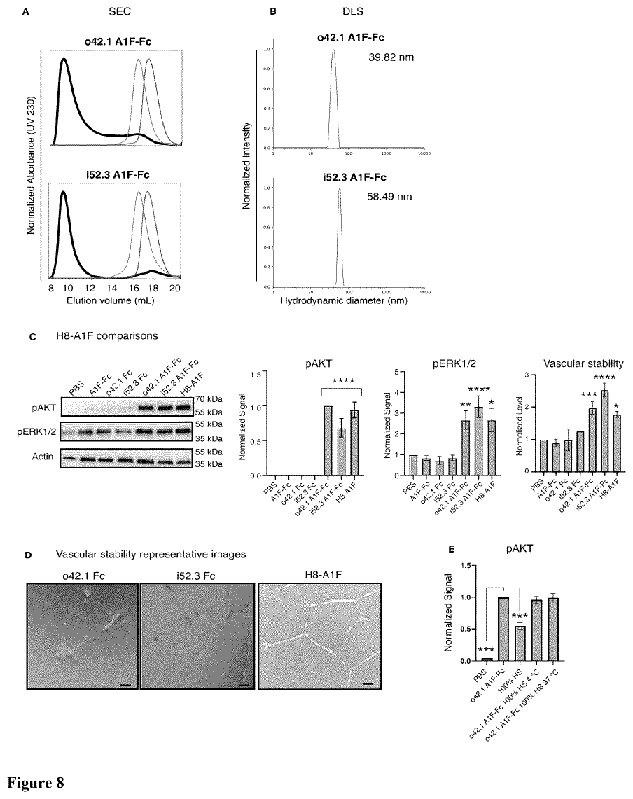

Certain receptor tyrosine kinases (RTKs), such as the Angiopoietin-1 receptor (Tie2), activate downstream signaling cascades when clustered (31, 32). Scaffolding the F-domain from angiopoietin-1 (AIF) onto nanoparticles induces phosphorylation of AKT and ERK, enhances cell migration and tube formation in vitro, and improves wound healing after injury in vivo (32). Therapeutics with these activities could be useful in treating conditions characterized by cell death and inflammation, such as sepsis and acute respiratory distress syndrome (ARDS). To determine whether the AbC platform could be used to generate such agonists, we assembled o42.1 and i52.3 AbCs with Fc fusions to A1F (FIG. 4g, FIG. 4a-b). The octahedral and icosahedral A1F-AbCs, but not Fc-only controls or free Fc-Ang1F, significantly increased AKT and ERK1/2 phosphory lation above baseline (FIG. 4h-i) and enhanced cell migration and vascular stability (FIG. 4j-k, FIG. 8c-d). These results show that the AbCs are more potent inducers of angiogenesis than free A1F-Fc, and as the components can be readily produced in large quantities, they are promising therapeutic candidates.

### α-CD40 Nanocages Activate B Cells

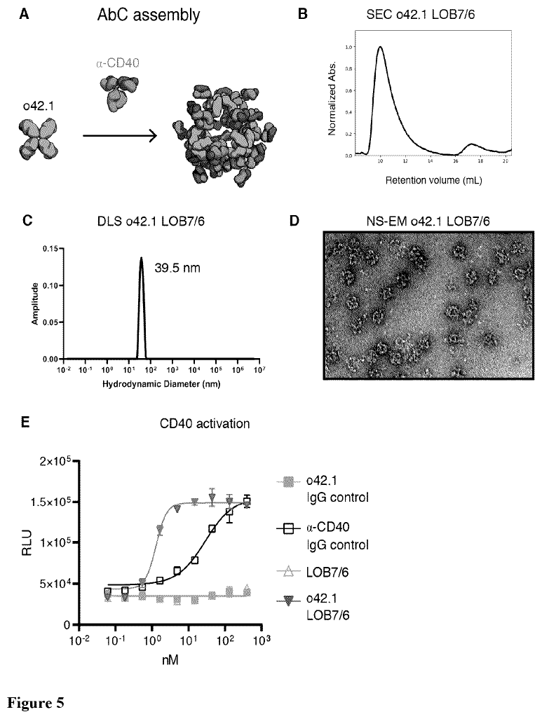

CD40, a TNFR superfamily member expressed on antigen presenting dendritic cells and B cells, is cross-linked by trimeric CD40 ligand (CD40L or CD154) on T cells, leading to signaling and cell proliferation (33, 34). Non-agonistic α-CD40 antibodies can be converted to agonists by adding cross-linkers such as FcγRIIb-expressing Chinese Hamster Ovary (CHO) cells (33). We investigated whether assembling a non-agonist α-CD40 antibody (LOB7/6) into nanocages could substitute for the need for cell surface presentation. Octahedral AbCs were assembled with LOB7/6 IgG; SEC, dynamic light scattering (DLS), and NS-EM (FIG. 5a-d) characterization showed these to be monodisperse with the expected octahedral shape. The octahedral α-CD40 LOB7/6 AbCs were found to induce robust CD40) activation in CD40-expressing reporter CHO cells (J215A, Promega), at concentrations hundredfold less than a control activating α-CD40 antibody (Promega), while no activation was observed for the free LOB7/6 antibody or octahedral AbC formed with non-CD40 binding IgG (FIG. 5e, Table 7). This demonstrates that nanocage assembly converts the non-agonist α-CD40 mAb into a CD40 pathway agonist.

TABLE 7
EC50s from CD40 activation experiments. EC50 values were
interpolated from the response curves determined using
the log(agonist) vs. response--Variable slope
(four parameters) fit using Graphpad Prism ™ Software

| | EC50 log(μM) | 95% CI log(μM) |
|---|---|---|
| o42.1 IgG control | −1.422 | Not found |
| α-CD40 | 1.466 | 1.247 to 1.833 |
| LOB7/6 | −1.471 | Not found |
| o42.1 LOB7/6 | 0.1134 | −0.001058 to 0.2037 |

TABLE 8
List of antibodies formed into cages as verified by at
minimum size exclusion chromatography. Successfully formed
cages (by SEC) listed by the antibody target reactivity,
antibody species and isotype, and designs used.

| | | Designs (validated by | |
|---|---|---|---|
| Ab reactivity | Ab subclass | SEC at minimum) | Comments |
| α-CD4 | mIgG2b | o42.1 | OKT4 |
| α-CD40 | mIgG2a or | o42.1 | LOB7/6 or 82111 |
| | mIgG2b | | (respectively) |
| α-DR5 | hIgG1 | d2.3, d2.4, d2.7, t32.4, | conatumumab |
| (human) | | t32.8, o42.1, i52.3, i52.6 | |
| α-DR5 | Armenian | t32.4, o42.1 | MD5-1 |
| (mouse) | hamster IgG | | |
| α-EGFR | hIgG1 | mIgG2b | cetuximab |
| α-LRP6 | hIgG1 | t32.4, o42.1 | YW210.09 |
| α-RSV F | hIgG1 | d2.3, d2.4, d2.7, t32.4, | mpe8 |
| | | t32.8, o42.1, i52.3, i52.6 | |
| Non-specific | Rabbit IgG | d2.4, o42.1 | Rabbit |
| | | | serum IgG |

TABLE 9
List of Fc-fusions formed into cages as verified by at minimum
size exclusion chromatography. Successfully formed cages
(by SEC) listed by the ligand that was fused to Fc, the
Fc sequence species and isotype, and designs used

| | | | |
|---|---|---|---|
| | Fc | Designs (validated by | |
| Fc-fusion ligand | subclass | SEC at minimum) | Comments |

| | | |
|---|---|---|
| Angiopoietin-1 F-domain | hIgG1 | d2.4, t32.4, t32.8, |
| | | o42.1, i52.3 |
| Angiotensin-converting | hIgG1 | o42.1 |
| enzyme 2 (ACE2) | | |
| CD80 | hIgG1 | o42.1 |
| mRuby2 | hIgG1 | d2.4, t32.4, t32.8, |
| | | o42.1, i52.3 |
| sfGFP | hIgG1 | d2.4, t32.4, t32.8, |
| | | o42.1, i52.3 |
| VEGF-a | hIgG1 | t32.4, o42.1 |
| VEGF-c | hIgG1 | t32.4, o42.1 |

### Discussion

Our approach goes beyond previous computational design efforts to create functional nanomaterials by integrating form and function; our AbCs employ antibodies as both structural and functional components. By fashioning designed antibody-binding, cage-forming oligomers through rigid helical fusion, a wide range of geometries and orientations can be achieved. This design strategy can be generalized to incorporate other homo-oligomers of interest into cage-like architectures. For example, nanocages could be assembled with viral glycoprotein antigens using components terminating in helical antigen-binding proteins, or from symmetric enzymes with exposed helices available for fusion to maximize proximity of active sites working on successive reactions. The AbCs offer considerable advantages in modularity compared to previous fusion of functional domain approaches; any of the thousands of known antibodies with sufficient protein A binding can be simply mixed with the appropriate design to drive formation of the desired symmetric assembly, and we have demonstrated this principle using multiple different IgGs and Fc-fusions (Tables 9-10). EM and SEC demonstrate monodispersity comparable to IgM and not (to our knowledge) attained by any other antibody-protein nanoparticle formulations.

AbCs show considerable promise as signaling pathway agonists. Assembly of antibodies against RTK- and TNFR-family cell-surface receptors into AbCs led to activation of diverse downstream signaling pathways involved in cell death, proliferation, and differentiation. While antibody-mediated clustering has been previously found to activate signaling pathways (11, 27, 33), our approach has the advantage of much higher structural homogeneity, allowing more precise tuning of phenotypic effects and more controlled formulation. AbCs also enhanced antibody-mediated viral neutralization. There are exciting applications to targeted delivery, as the icosahedral AbCs have substantial internal volume (around 15,000 nm3, based on an estimated interior radius of 15.5 nm) that could be used to package nucleic acid or protein cargo, and achieving different target specificity in principle is as simple as swapping one antibody for another. We anticipate that the AbCs developed here, coupled with the very large repertoire of existing antibodies, will be broadly useful across a wide range of applications in biomedicine.

### REFERENCES AND NOTES

1. R.-M. Lu, Y.-C. Hwang, I.-J. Liu, C.-C. Lee, H.-Z. Tsai, H.-J. Li, H.-C. Wu, Development of therapeutic antibodies for the treatment of diseases. J. Biomed. Sci. 27, 1 (2020).
2. H. J. Kang, Y. J. Kang, Y.-M. Lee, H.-H. Shin, S. J. Chung, S. Kang, Developing an antibody-binding protein cage as a molecular recognition drug modular nanoplatform. Biomaterials. 33, 5423-5430 (2012).
3. H. Kim, Y. J. Kang, J. Min, H. Choi, S. Kang, Development of an antibody-binding modular nanoplatform for antibody-guided targeted cell imaging and delivery. RSC Advances. 6 (2016), pp. 19208-19213.
4. A. M. Cuesta, N. Sainz-Pastor, J. Bonet, B. Oliva, L. Alvarez-Vallina, Multivalent antibodies: when design surpasses evolution. Trends Biotechnol. 28, 355-362 (2010).
5. N. Nuñez-Prado, M. Compte, S. Harwood, A. Álvarez-Méndez, S. Lykkemark, L. Sanz, L. Álvarez-Vallina, The coming of age of engineered multivalent antibodies. Drug Discovery Today. 20 (2015), pp. 588-594.
6. A. Miller, S. Carr, T. Rabbitts, H. Ali, Multimeric antibodies with increased valency surpassing functional affinity and potency thresholds using novel formats. MAbs. 12, 1752529 (2020).
7. M. Klement, J. Zheng, C. Liu, H.-L. Tan, V. V. T. Wong, A. B.-H. Choo, D.-Y. Lee, D. S.-W. Ow, Antibody engineering of a cytotoxic monoclonal antibody 84 against human embryonic stem cells: Investigating the effects of multivalency on cytotoxicity. Journal of Biotechnology. 243 (2017), pp. 29-37.
8. N. S. Laursen, R. H. E. Friesen, X. Zhu, M. Jongeneelen, S. Blokland, J. Vermond, A. van Eijgen, C. Tang, H, van Diepen, G. Obmolova, M, van der Neut Kolfschoten, D. Zuijdgeest, R. Straetemans, R. M. B. Hoffman, T. Nieusma, J. Pallesen, H. L. Turner, S. M. Bernard, A. B. Ward, J. Luo, L. L. M. Poon, A. P. Tretiakova, J. M. Wilson, M. P. Limberis, R. Vogels, B. Brandenburg, J. A. Kolkman, I. A. Wilson, Universal protection against influenza infection by a multidomain antibody to influenza hemagglutinin. Science. 362, 598-602 (2018).
9. O. Seifert, A. Plappert, S. Fellermeier, M. Siegemund, K. Pfizenmaier, R. E. Kontermann, Tetravalent Antibody-scTRAIL Fusion Proteins with Improved Properties. Molecular Cancer Therapeutics. 13 (2014), pp. 101-111.
10. M. Siegemund, F. Schneider, M. Hutt, O. Seifert, I. Müller, D. Kulms, K. Pfizenmaier, R. E. Kontermann, IgG-single-chain TRAIL fusion proteins for tumour therapy. Sci. Rep. 8, 7808 (2018).
11. R. S. Riley, E. S. Day, Frizzled7 Antibody-Functionalized Nanoshells Enable Multivalent Binding for Wnt Signaling Inhibition in Triple Negative Breast Cancer Cells. Small. 13 (2017), doi: 10.1002/smll.201700544.
12. E. Hiramoto, A. Tsutsumi, R. Suzuki, S. Matsuoka, S. Arai, M. Kikkawa, T. Miyazaki, The IgM pentamer is an asymmetric pentagon with an open groove that binds the AIM protein. Sci Adv. 4, eaau1199 (2018).
13. N. P. King, W. Sheffler, M. R. Sawaya, B. S. Vollmar, J. P. Sumida, I. André, T. Gonen, T. O. Yeates, D. Baker, Computational design of self-assembling protein nanomaterials with atomic level accuracy. Science. 336, 1171-1174 (2012).
14. J. B. Bale, S. Gonen, Y. Liu, W. Sheffler, D. Ellis, C. Thomas, D. Cascio, T. O. Yeates, T. Gonen, N. P. King, D. Baker, Accurate design of megadalton-scale two-component icosahedral protein complexes. Science. 353, 389-394 (2016).
15. G. Ueda, A. Antanasijevic, J. A. Fallas, W. Sheffler, J. Copps, D. Ellis, G. Hutchinson, A. Moyer, A. Yasmeen, Y. Tsy bovsky, Y.-J. Park, M. J. Bick, B. Sankaran, R. A. Gillespie, P. J. M. Brouwer, P. H. Zwart, D. Veesler, M. Kanekiyo, B. S. Graham, R. Sanders, J. P. Moore, P. J. Klasse, A. B. Ward, N. King, D. Baker, Tailored Design of Protein Nanoparticle Scaffolds for Multivalent Presentation of Viral Glycoprotein Antigens, doi: 10.1101/2020.01.29.923862.
16. M. Graille, E. A. Stura, A. L. Corper, B. J. Sutton, M. J. Taussig, J.-B. Charbonnier, G. J. Silverman, Crystal structure of a Staphylococcus aureus protein A domain complexed with the Fab fragment of a human IgM antibody: Structural basis for recognition of B-cell receptors and superantigen activity. Proceedings of the National Academy of Sciences. 97 (2000), pp. 5399-5404.
17. T. J. Brunette, F. Parmeggiani, P.-S. Huang, G. Bhabha, D. C. Ekiert, S. E. Tsutakawa, G. L. Hura, J. A. Tainer, D. Baker, Exploring the repeat protein universe through computational protein design. Nature. 528, 580-584 (2015).
18. J. A. Fallas, G. Ueda, W. Sheffler, V. Nguyen, D. E. McNamara, B. Sankaran, J. H. Pereira, F. Parmeggiani, T. J. Brunette, D. Cascio, T. R. Yeates, P. Zwart, D. Baker, Computational design of self-assembling cyclic protein homo-oligomers. Nat. Chem. 9, 353-360 (2017).
19. P.-S. Huang, G. Oberdorfer, C. Xu, X. Y. Pei, B. L. Nannenga, J. M. Rogers, F. DiMaio, T. Gonen, B. Luisi, D. Baker, High thermodynamic stability of parametrically designed helical bundles. Science. 346, 481-485 (2014).
20. I. Vulovic, Q. Yao, Y.-J. Park, A. Courbet, A. Norris, F. Busch, A. Sahasrabuddhe, H. Merten, D. D. Sahtoe, G. Ueda, J. A. Fallas, S. J. Weaver, Y. Hsia, R. A. Langan, A. Plückthun, V. H. Wysocki, D. Veesler, G. J. Jensen, D. Baker, Generation of ordered protein assemblies using rigid three-body fusion,, doi: 10.1101/2020.07.18.210294.
21. Y. Hsia, R. Mout, W. Sheffler, N. I. Edman, I. Vulovic, Y.-J. Park, R. L. Redler, M. J. Bick, A. K. Bera, A. Courbet, A. Kang, T. J. Brunette, U. Nattermann, E. Tsai, A. Saleem, C. M. Chow, D. Ekiert, G. Bhabha, D. Veesler, D. Baker, Hierarchical design of multi-scale protein complexes by combinatorial assembly of oligomeric helical bundle and repeat protein building blocks (2020).
22. T. O. Yeates, Y. Liu, J. Laniado, The design of symmetric protein nanomaterials comes of age in theory and practice. Curr. Opin. Struct. Biol. 39, 134-143 (2016).
23. B. M. Baynes, D. I. C. Wang, B. L. Trout, Role of arginine in the stabilization of proteins against aggregation. Biochemistry. 44, 4919-4925 (2005).
24. D. Schneidman-Duhovny, M. Hammel, J. A. Tainer, A. Sali, Accurate SAXS profile computation and its assessment by contrast variation experiments. Biophys. J. 105, 962-974 (2013).
25. J. L. Taupin, P. Legembre, J. Bitard, S. Daburon, V. Pitard, F. Blanchard, L. Duplomb, A. Godard, Y. Jacques, J. F. Moreau, Identification of agonistic and antagonistic antibodies against gp190, the leukemia inhibitory factor receptor, reveals distinct roles for its two cytokine-binding domains. J. Biol. Chem. 276, 47975-47981 (2001).
26. K. Mohan, G. Ueda, A. R. Kim, K. M. Jude, J. A. Fallas, Y. Guo, M. Hafer, Y. Miao, R. A. Saxton, J. Piehler, V. G. Sankaran, D. Baker, K. C. Garcia, Topological control of cytokine receptor signaling induces differential effects in hematopoiesis. Science. 364 (2019), doi:10.1126/science.aav7532.
27. J. D. Graves, J. J. Kordich, T.-H. Huang, J. Piasecki, T. L. Bush, T. Sullivan, I. N. Foltz, W. Chang, H. Douangpanya, T. Dang, J. W. O'Neill, R. Mallari, X. Zhao, D. G. Branstetter, J. M. Rossi, A. M. Long, X. Huang, P. M. Holland, Apo2L/TRAIL and the death receptor 5 agonist antibody AMG 655 cooperate to promote receptor clustering and antitumor activity. Cancer Cell. 26, 177-189 (2014).
28. J. Naval, D, de Miguel, A. Gallego-Lleyda, A. Anel, L. Martinez-Lostao, Importance of TRAIL Molecular Anatomy in Receptor Oligomerization and Signaling. Implications for Cancer Therapy. Cancers. 11 (2019), doi:10.3390/cancers 11040444.
29. D, de Miguel, J. Lemke, A. Anel, H. Walczak, L. Martinez-Lostao, Onto better TRAILs for cancer treatment. Cell Death Differ. 23, 733-747 (2016).
30. M. H. Tuthill, A. Montinaro, J. Zinngrebe, K. Prieske, P. Draber, S. Prieske, T. Newsom-Davis, S, von Karstedt, J. Graves, H. Walczak, TRAIL-R2-specific antibodies and recombinant TRAIL can synergise to kill cancer cells. Oncogene. 34, 2138-2144 (2015).
31. V.-M. Leppänen, P. Saharinen, K. Alitalo, Structural basis of Tie2 activation and Tie2/Tie1 heterodimerization. Proc. Natl. Acad. Sci. U. S. A. 114, 4376-4381 (2017).
32. Y.-T. Zhao, J. A. Fallas, S. Saini, G. Ueda, L. Somasundaram, Z. Zhou, I. Xavier, D. Ehnes, C. Xu, L. Carter, S. Wrenn, J. Mathieu, D. L. Sellers, D. Baker, H. Ruohola-Baker, F-domain valency determines outcome of signaling through the Angiopoietin pathway (2020).
33. R. S. Kornbluth, M. Stempniak, G. W. Stone, Design of CD40 agonists and their use in growing B cells for cancer immunotherapy. Int. Rev. Immunol. 31, 279-288 (2012).
34. R. H. Vonderheide, M. J. Glennie, Agonistic CD40 Antibodies and Cancer Therapy. Clinical Cancer Research. 19 (2013), pp. 1035-1043.
35. E. R. Steenblock, S. H. Wrzesinski, R. A. Flavell, T. M. Fahmy, Antigen presentation on artificial acellular substrates: modular systems for flexible, adaptable immunotherapy. Expert Opin. Biol. Ther. 9, 451-464 (2009).
36. J. V. Kim, J.-B. Latouche, I. Rivière, M. Sadelain, The ABCs of artificial antigen presentation. Nat. Biotechnol. 22, 403-410 (2004).
39. E. E. Idusogie, L. G. Presta, H. Gazzano-Santoro, K. Totpal, P. Y. Wong, M. Ultsch, Y. G. Meng, M. G. Mulkerrin, Mapping of the Clq binding site on rituxan, a chimeric antibody with a human IgG1 Fc. J. Immunol. 164, 4178-4184 (2000).
40. F. DiMaio, N. Echols, J. J. Headd, T. C. Terwilliger, P. D. Adams, D. Baker, Improved low-resolution crystallographic refinement with Phenix and Rosetta. Nature Methods. 10 (2013), pp. 1102-1104.
41. F. W. Studier, F. William Studier, Protein production by auto-induction in high-density shaking cultures. Protein Expression and Purification. 41 (2005), pp. 207-234.
42. J. K. Leman, B. D. Weitzner, S. M. Lewis, J. Adolf-Bryfogle, N. Alam, R. F. Alford, M. Aprahamian, D. Baker, K. A. Barlow, P. Barth, B. Basanta, B. J. Bender, K. Blacklock, J. Bonet, S. E. Boyken, P. Bradley, C. Bystroff, P. Conway, S. Cooper, B. E. Correia. B. Coventry, R. Das, R. M. De Jong, F. DiMaio, L. Dsilva, R. Dunbrack, A. S. Ford, B. Frenz, D. Y. Fu. C. Geniesse. L. Goldschmidt, R. Gowthaman, J. J. Gray, D. Gront, S. Guffy, S. Horowitz, P.-S. Huang. T. Huber, T. M. Jacobs, J. R. Jeliazkov, D. K. Johnson, K. Kappel, J. Karanicolas, H. Khakzad, K. R. Khar, S. D. Khare, F. Khatib, A. Khramushin, I. C. King, R. Kleffner, B. Koepnick, T. Kortemme, G. Kuenze. B. Kuhlman, D. Kuroda, J. W. Labonte, J. K. Lai, G. Lapidoth, A. Leaver-Fay, S. Lindert, T. Linsky, N. London, J. H. Lubin, S. Lyskov, J. Maguire, L. Malmström, E. Marcos, O. Marcu, N. A. Marze, J. Meiler, R. Moretti, V. K. Mulligan, S. Nerli, C. Norn, S. Ó'Conchúir, N. Ollikainen, S. Ovchinnikov, M. S. Pacella, X. Pan, H. Park, R. E. Pavlovicz, M. Pethe, B. G. Pierce, K. B. Pilla, B. Raveh, P. Douglas Renfrew, S. S. Roy Burman, A. Rubenstein, M. F. Sauer, A. Scheck, W. Schief, O. Schueler-Furman, Y. Sedan, A. M. Sevy, N. G. Sgourakis, L. Shi, J. B. Siegel, D.-A. Silva, S. Smith, Y. Song, A. Stein, M. Szegedy, F. D. Teets, S. B. Thyme, R. Y.-R. Wang, A. Watkins, L. Zimmerman, R. Bonneau, Macromolecular modeling and design in Rosetta: recent methods and frameworks. Nat. Methods. 17, 665-680 (2020).
43. L. Jendeberg, P. Nilsson, A. Larsson, P. Denker, M. Uhlén, B. Nilsson, P.-Å. Nygren, Engineering of Fc1 and Fc3 from human immunoglobulin G to analyse subclass specificity for staphylococcal protein A. Journal of Immunological Methods. 201 (1997), pp. 25-34.
44. D. Corti, S. Bianchi, F. Vanzetta, A. Minola, L. Perez, G. Agatic, B. Guarino, C. Silacci, J. Marcandalli, B. J. Marsland, A. Piralla, E. Percivalle, F. Sallusto, F. Baldanti, A. Lanzavecchia, Cross-neutralization of four paramyxoviruses by a human monoclonal antibody. Nature. 501, 439-443 (2013).
45. K. N. Dyer, M. Hammel, R. P. Rambo, S. E. Tsutakawa, I. Rodic, S. Classen, J. A. Tainer, G. L. Hura, High-throughput SAXS for the characterization of biomolecules in solution: a practical approach. Methods Mol. Biol. 1091, 245-258 (2014).

### Materials and Methods

### Computational Design and Testing of Fc-Binder Helical Repeat Protein (DHR79-FcB)

The crystal structure of the B-domain from S. aureus protein A in complex with Fc fragment (PDB ID: 1L6X) was relaxed with structure factors using Phenix Rosetta™ (39, 40). Briefly, the RosettaScripts™ MotifGraft mover was used to assess suitable solutions to insertions of the protein A binding motif extracted from 1L6X into a previously reported designed helical repeat protein (DHR79) (17). Specifically, a minimal protein A binding motif was manually defined and extracted and used as a template for full backbone alignment of DHR79 while retaining user-specified hotspot residues that interact with the Fc domain in the crystal structure at the Fc/DHR interface and retaining native DHR residues in all other positions. The MotifGraft alignment was followed by 5 iterations of FastDesign and 5 iterations of FastRelax in which the DHR side chain and backbone rotamers were allowed to move while the Fc context was completely fixed. The best designs were selected based on a list of heuristic filter values. See supplementary materials for the full XML file used during design. FIG. 61a shows the design model of DHR79-FcB.

Designs were initially assessed via yeast surface display binding to biotinylated Fc protein. Upon confirmation of a qualitative binding signal, the design was closed into a pET29b expression vector with a C-terminal His-tag. The protein was expressed in BL21 DE3 in autoinduction medium (10 mL 50×M, 10 mL 50×5052, 480 mL almost TB, 1×chloramphenicol, 1×kanamycin) for 20 hours at 27° C. at 225 rpm (41). Cells were resuspended in lysis buffer (20 mM Tris, 300 mM NaCl, 30 mM imidazole, 1 mM PMSF, 5% glycerol (v/v), pH 8.0) and lysed using a microfluidizer at 18000 PSI. Soluble fractions were separated via centrifugation at 24.000×g. IMAC with Ni-NTA batch resin was used for initial purification; briefly, nickel-nitrilotriacetic acid (Ni-NTA) resin was equilibrated with binding buffer (20 mM Tris, 300 mM NaCl, 30 mM imidazole, pH 8.0), soluble lysate was poured over the columns, columns were washed with 20 column volumes (CVs) of binding buffer, and eluted with 5 CVs of elution buffer (20 mM Tris, 300 mM NaCl, 500 mM imidazole, pH 8.0). Size exclusion chromatography (SEC) with a Superdex 200 column was used as the polishing step (FIG. 6b). SEC buffer was 20 mM Tris/HCl pH 7.4, 150 mM NaCl.

Affinity of DHR79-FcB to biotinylated IgG1 and biotinylated Fc protein was assessed using Octet™ Biolayer Interferometry (BLI). DHR79-FcB exhibits a 71.7 nM affinity to IgG1 (full antibody) and a 113 nM affinity to the IgG1 Fc protein (FIG. 6c).

### Computational Design of Antibody Nanocages

Input pdb files were compiled to use as building blocks for the generation of antibody cages. For the protein A binder model, the Domain D from Staphylococcus aureus Protein A (PDB ID 1DEE) was aligned to the B-domain of protein A bound to Fc (PDB ID 1L6X) (16, 39). The other Fc-binding design structure, where protein A was grafted onto a helical repeat protein, was also modeled with Fc from 1L6X. PDB file models for monomeric helical repeat protein linkers (42) and cyclic oligomers (2 C2s, 3 C3s, 1 C4, and 2 C5s) that had at least been validated via SAXS were compiled from previous work from our lab (17-19). Building block models were manually inspected to determine which amino acids were suitable for making fusions without disrupting existing protein-protein interfaces.

These building blocks were used as inputs, along with the specified geometry and fusion orientation, into the alpha helical fusion software (Supplementary Text for a description on how to operate WORMS) (20, 21). Fusions were made by overlapping helical segments at all possible allowed amino acid sites. Fusions are then evaluated for deviation for which the cyclic symmetry axes intersect according to the geometric criteria: D2, T32, O32,O42, I32, and I52 intersection angles are 45.0°, 54.7°, 35.3°, 45.0°, 20.9°, and 31.7°, respectively (22) with angular and distance tolerances of at most 5.7° and 0.5 Å respectively. Post-fusion .pdb files were manually filtered to ensure that the N-termini of the Fc domains are facing outwards from the cage, so that the Fabs of an IgG would be external to the cage surface. Sequence design was performed using Rosetta™ symmetric sequence design (SymPackRotamersMover in RosettaScripts™) on residues at and around the fusion junctions (42), with a focus on maintaining as many of the native residues as possible. Residues were redesigned if they clashed with other residues, or if their chemical environment was changed after fusion (e.g, previously-core facing residues were now solvent-exposed). Index residue selectors were used to prevent design at Fc residue positions.

Structural Characterization of Antibody Nanocages

Genes were codon optimized for bacterial expression of each designed antibody-nanocage forming oligomers, with a C-terminal glycine/serine linker and 6× C-terminal histidine tag appended. Synthetic genes were cloned into pet29b+ vectors between NdeI and XhoI restriction sites; the plasmid contains a kanamycin-resistant gene and T7 promoter for protein expression. Plasmids were transformed into chemically competent Lemo21 (DE3) E. coli bacteria using a 15-second heat shock procedure as described by the manufacturer (New England Biolabs). Transformed cells were added to auto-induction expression media, as described above, and incubated for 16 hours at 37° C. and 200 rpm shaking (41). Cells were pelleted by centrifugation at 4000×g and resuspended in lysis buffer (150 mM NaCl, 25 mM Tris-HCl, pH 8.0, added protease inhibitor and DNAse). Sonication was used to lyse the cells at 85% amplitude, with 15 second on/off cycles for a total of 2 minutes of sonication time. Soluble material was separated by centrifugation at 16000×g. IMAC was used to separate out the His-tagged protein in the soluble fraction as described above. IMAC elutions were concentrated to approximately 1 mL using 10K MWCO spin concentrators, filtered through a 0.22 uM spin filter, and run over SEC as a final polishing step (SEC running buffer: 150 mM NaCl, 25 mM Tris-HCl, pH 8.0).

Designs that produced monodisperse SEC peaks around their expected retention volume were combined with Fc from human IgG1. Fc was produced recombinantly either using standard methods for expression in HEK293T cells or in E. coli (43). Cage components were incubated at 4° C. for at minimum 30 minutes. 100 mM L-arginine was added during the assembly to AbCs formed with the i52.6 design, as this was observed to maximize the formation of the designed AbC i52.6 and minimize the formation of visible “crashed out” aggregates (23). Fc-binding and cage formation were confirmed via SEC; earlier shifts in retention time (compared to either component run alone) show the formation of a larger structure. NS-EM was used as previously described to confirm the structures of designs that passed these steps.

For confirming AbC structures with intact IgGs, human IgG1 (hIgG1) was combined with AbC-forming designs following the same protocol for making Fc cages. This assembly procedure was also followed for all IgG or Fc-fusion AbCs reported hereafter. The data in FIG. 2d-e shows AbCs formed with the α-DR5 antibody AMG-655 (23) for the following designs: d2.3, d2.4, d2.7, t32.4, o42.1, and i52.3. The data for t32.8 and i52.6 designs shown in FIG. 2d-e is from AbCs formed with the hIgG1 antibody mpe8 (44). Tables 9 and 10 show the list of IgGs and Fc fusions that have been formed into AbCs.

Dynamic light scattering measurements (DLS) were performed using the default Sizing and Polydispersity method on the UNcle™ (Unchained Labs). 8.8 μL of AbCs were pipetted into the provided class cuvettes. DLS measurements were run in triplicate at 25° C. with an incubation time of 1 second; results were averaged across runs and plotted using Graphpad Prism. The estimated hydrodynamic diameter is listed next to all DLS peaks shown below.

### NS-EM analysis of Fc and IgG AbCs

For all samples except o42.1 Fc and i52.3 Fc, 3.0 μL of each SEC-purified sample between 0.008-0.014 mg/mL in TBS pH 8.0 was applied onto a 400-mesh or 200-mesh Cu grid glow-discharged carbon-coated copper grids for 20 seconds, followed by 2× application of 3.0 μL 2% nano-W stain. Micrographs were recorded using Leginon software on a 120 kV FEI Tecnai G2 Spirit™ with a Gatan Ultrascan™ 4000 4k×4k CCD camera at 67,000 nominal magnification (pixel size 1.6 Å/pixel) or 52,000 nominal magnification (pixel size 2.07 Å) at a defocus range of 1.5-2.5 μm. Particles were picked either with DoGPicker or cisTEM; both are reference-free pickers. Contrast-transfer function was estimated using GCTF or cisTEM. 2D class averages were generated in cryoSPARC or in cisTEM. Reference-free ab initio 3D reconstruction of selected 2D class averages from each dataset was performed in cryoSPARC or in cisTEM (Table 11).

TABLE 10
Details on data acquisition and data processing of different nanocages samples.

| Sample | | Voltage | | Pixel size | Particle | CTF | 2D class | 3D |
|---|---|---|---|---|---|---|---|---|
| name | Stain | (kV) | Magnification | (Å/pixiel) | picking | estimation | averages | reconstruction |
| d2.3 Fc | UF | 120 | 67,000 | 1.6 | cisTEM | cisTEM | cisTEM | cisTEM |
| d2.4 Fc | UF | 120 | 67,000 | 1.6 | DoG | GCTF | cryoSPARC | cryoSPARC |
| | | | | | picker | | | |
| d2.7 Fc | nano-W | 120 | 67,000 | 1.6 | cisTEM | cisTEM | cisTEM | cisTEM |
| t32.4 Fc | nano-W | 120 | 67,000 | 1.6 | cisTEM | cisTEM | cisTEM | cisTEM |
| t32.8 Fc | nano-W | 120 | 67,000 | 1.6 | cisTEM | cisTEM | cisTEM | cisTEM |
| o42.1 Fc | cryo | 200 | 36,000 | 1.16 | Manual | GCTF | cryoSPARC | cryoSPARC |
| | | | | | picking | | | |
| i52.3 Fc | cryo | 200 | 36,000 | 1.16 | Manual | GCTF | cryoSPARC | cryoSPARC |
| | | | | | picking | | | |
| i52.6 Fc | nano-W | 120 | 52,000 | 2.07 | cisTEM | cisTEM | cisTEM | cisTEM |
| d2.3 hIgG1 | UF | 120 | 67,000 | 1.6 | cisTEM | cisTEM | cisTEM | N/A |
| d2.4 hIgG1 | UF | 120 | 67,000 | 1.6 | cisTEM | cisTEM | cisTEM | N/A |
| d2.7 hIgG1 | nano-W | 120 | 67,000 | 1.6 | cisTEM | cisTEM | cisTEM | N/A |
| t32.4 hIgG1 | nano-W | 120 | 67,000 | 1.6 | cisTEM | cisTEM | cisTEM | N/A |
| t32.8 hIgG1 | nano-W | 120 | 67,000 | 1.6 | cisTEM | cisTEM | cisTEM | N/A |
| o42.1 hIgG1 | UF | 120 | 67,000 | 1.6 | DoG | GCTF | cryoSPARC | N/A |
| | | | | | picker | | | |
| i52.3 hIgG1 | nano-W | 120 | 53,000 | 2.07 | cisTEM | cisTEM | cisTEM | N/A |
| i52.6 hIgG1 | nano-W | 120 | 52,000 | 2.07 | cisTEM | cisTEM | cryoSPARC | N/A |
| D3-08 Fc | nano-W | 120 | 57,000 | 2.52 | cisTEM | cisTEM | cisTEM | cisTEM |
| D3-36 Fc | nano-W | 120 | 57,000 | 2.52 | cisTEM | cisTEM | cisTEM | cisTEM |

### Cryo-EM Analysis of o42.1 and i52.3 AbCs

3.0 μL of i52.3 Fc sample at 0.8 mg/mL in TBS pH 8.0 with 100 mM Arginine was applied onto C-flat 1.2 μm glow-discharged copper grids. Grids were then plunge-frozen in liquid ethane, cooled with liquid nitrogen using and FEI MK4 Vitrobot with a 6 second blotting time and 0 force. The blotting process took place inside the Vitrobot chamber at 20° C. and 100% humidity. Data acquisition was performed with the Leginon data collection software on an FEI Talos electron microscope at 200 kV and a Gatan K2 Summit camera. The nominal magnification was 36,000× with a pixel size of 1.16 Å/pixel. The dose rate was adjusted to 8 counts/pixel/s. Each movie was acquired in counting mode fractionated in 50 frames of 200 ms/frame. Frame alignment was performed with MotionCorr2. Particles were manually picked within the Appion interface. Defocus parameters were estimated with GCTF. Reference-free 2D classification with cryoSPARC was used to select a subset of particles for Ab-Initio 3D reconstruction function in cryoSPARC.

A summary of data acquisition and processing is provided in Table 11.

### DR5 and A1F-Fc Experiments

### Cell Culture

Colorectal adenocarcinoma cell line-Colo205, and renal cell carcinoma cell line RCC4 were obtained from ATCC. Primary kidney tubular epithelial cells RAM009 were a gift from Dr. Akilesh (University of Washington). Colo205 cells were grown in RPMI1640 medium with 10% Fetal Bovine Serum (FBS) and penicillin/streptomyocin. RCC4 cells were grown in Dulbecco's Modified Eagle's Medium with 10% FBS and penicillin/streptomyocin. RAM009 were grown in RPMI with 10% FBS, ITS-supplement, penicillin/streptomyocin and Non Essential Amino Acids (NEAA). All cell lines were maintained at 37° C. in a humidified atmosphere containing 5% CO2.

Human Umbilical Vein Endothelial Cells (HUVECs, Lonza, Germany, catalog # C2519AS) were grown on 0.1% gelatin-coated 35 mm cell culture dish in EGM2 media. Briefly, EGM2 consist of 20% Fetal Bovine Serum, 1% penicillin-streptomycin, 1% Glutamax (Gibco, catalog #35050061), 1% endothelial cell growth factor (31), 1 mM sodium pyruvate, 7.5 mM HEPES, 0.08 mg/mL heparin, 0.01% amphotericin B, a mixture of 1× RPMI 1640 with and without glucose to reach 5.6 mM glucose concentration in the final volume. Media was filtered through a 0.45-micrometer filter. HUVECs at passage 7 were utilized in Tie2 signaling and cell migration experiments. HUVECs at passage 6 were used in tube formation assay.

### Caspase 3/7 Glo Assay

Cells were passaged using trypsin and 20,000 cells/well were plated onto a 96-well white tissue culture plate and grown in appropriate media. Medium was changed the next day (100 μL/well) and cells were treated with either uncaged α-DR5 AMG655 antibody (150 nM), recombinant human TNF Related Apoptosis Inducing Ligand (rhTRAIL; 150 nM), Fc-only AbCs or α-DR5 AbCs (150 nM, 1.5 nM, 15 pM) and incubated at 37° C. for 24 hours. The following day 100 μL/well of caspase GLO™ reagent (Promega, USA) was added on top of the media and incubated for 2 hours at 37° C. Luminescence was then recorded using Perkin EnVision microplate reader (Perkin Elmer). Statistical comparisons were performed using Graphpad Prism™ (see Table 11 for full detail).

TABLE 11
Statistical information for DR5 experiments.

| Experiment | | | | | | Mean | | | Adjusted |
|---|---|---|---|---|---|---|---|---|---|
| (FIG.) | Condition | Concentration | | n | Test | compared to | α | Summary | P value |
| Caspase-3,7 | PBS | 15 | pM | 6 | 2way ANOVA with | N/A | N/A | N/A | N/A |
| RCC4 (4b) | | | | | post-hoc Dunnett | | | | |
| | | 1.5 | nM | 6 | 2way ANOVA with | 15 pM PBS | 0.05 | ns | 0.9996 |
| | | | | | post-hoc Dunnett | | | | |
| | | 150 | nM | 6 | 2way ANOVA with | 15 pM PBS | 0.05 | ns | 0.9994 |
| | | | | | post-hoc Dunnett | | | | |
| | TRAIL | 15 | pM | 6 | 2way ANOVA with | 15 pM PBS | 0.05 | ns | 0.9996 |
| | | | | | post-hoc Dunnett | | | | |
| | | 1.5 | nM | 6 | 2way ANOVA with | 15 pM PBS | 0.05 | ns | 0.9996 |
| | | | | | post-hoc Dunnett | | | | |
| | | 150 | nM | 6 | 2way ANOVA with | 15 pM PBS | 0.05 | ns | 0.9668 |
| | | | | | post-hoc Dunnett | | | | |
| | α-DR5 | 15 | pM | 6 | 2way ANOVA with | 15 pM PBS | 0.05 | ns | 0.9997 |
| | | | | | post-hoc Dunnett | | | | |
| | | 1.5 | nM | 6 | 2way ANOVA with | 15 pM PBS | 0.05 | ns | >0.9999 |
| | | | | | post-hoc Dunnett | | | | |
| | | 150 | nM | 6 | 2way ANOVA with | 15 pM PBS | 0.05 | ns | 0.2005 |
| | | | | | post-hoc Dunnett | | | | |
| | d2.4 | 15 | pM | 6 | 2way ANOVA with | 15 pM PBS | 0.05 | ns | 0.9995 |
| | α-DR5 | | | | post-hoc Dunnett | | | | |
| | | 1.5 | nM | 6 | 2way ANOVA with | 15 pM PBS | 0.05 | ns | 0.9988 |
| | | | | | post-hoc Dunnett | | | | |
| | | 150 | nM | 6 | 2way ANOVA with | 15 pM PBS | 0.05 | **** | <0.0001 |
| | | | | | post-hoc Dunnett | | | | |
| | t32.4 | 15 | pM | 6 | 2way ANOVA with | 15 pM PBS | 0.05 | ns | 0.9998 |
| | α-DR5 | | | | post-hoc Dunnett | | | | |
| | | 1.5 | nM | 6 | 2way ANOVA with | 15 pM PBS | 0.05 | **** | <0.0001 |
| | | | | | post-hoc Dunnett | | | | |
| | | 150 | nM | 6 | 2way ANOVA with | 15 pM PBS | 0.05 | **** | <0.0001 |
| | | | | | post-hoc Dunnett | | | | |
| | t32.8 | 15 | pM | 3 | 2way ANOVA with | 15 pM PBS | 0.05 | ns | 0.984 |
| | α-DR5 | | | | post-hoc Dunnett | | | | |
| | | 1.5 | nM | 3 | 2way ANOVA with | 15 pM PBS | 0.05 | ns | 0.6177 |
| | | | | | post-hoc Dunnett | | | | |
| | | 150 | nM | 3 | 2way ANOVA with | 15 pM PBS | 0.05 | **** | <0.0001 |
| | | | | | post-hoc Dunnett | | | | |
| | o42.1 | 15 | pM | 6 | 2way ANOVA with | 15 pM PBS | 0.05 | ns | 0.9991 |
| | α-DR5 | | | | post-hoc Dunnett | | | | |
| | | 1.5 | nM | 6 | 2way ANOVA with | 15 pM PBS | 0.05 | **** | <0.0001 |
| | | | | | post-hoc Dunnett | | | | |
| | | 150 | nM | 6 | 2way ANOVA with | 15 pM PBS | 0.05 | **** | <0.0001 |
| | | | | | post-hoc Dunnett | | | | |
| | i52.3 | 15 | pM | 3 | 2way ANOVA with | 15 pM PBS | 0.05 | ns | 0.9442 |
| | α-DR5 | | | | post-hoc Dunnett | | | | |
| | | 1.5 | nM | 3 | 2way ANOVA with | 15 pM PBS | 0.05 | ns | 0.8227 |
| | | | | | post-hoc Dunnett | | | | |
| | | 150 | nM | 3 | 2way ANOVA with | 15 pM PBS | 0.05 | **** | <0.0001 |
| | | | | | post-hoc Dunnett | | | | |
| Viability 4d | PBS | 150 | nM | 3 | 1way ANOVA with | N/A | N/A | N/A | N/A |
| RCC4 (4c) | | | | | post-hoc Dunnett | | | | |
| | TRAIL | 150 | nM | 3 | 1way ANOVA with | PBS | 0.05 | ns | 0.5207 |
| | | | | | post-hoc Dunnett | | | | |
| | α-DR5 | 150 | nM | 3 | 1way ANOVA with | PBS | 0.05 | ns | 0.9996 |
| | | | | | post-hoc Dunnett | | | | |
| | d2.4 | 150 | nM | 3 | 1way ANOVA with | PBS | 0.05 | * | 0.0238 |
| | α-DR5 | | | | post-hoc Dunnett | | | | |
| | t32.4 | 150 | nM | 3 | 1way ANOVA with | PBS | 0.05 | **** | <0.0001 |
| | α-DR5 | | | | post-hoc Dunnett | | | | |
| | t32.8 | 150 | nM | 3 | 1way ANOVA with | PBS | 0.05 | **** | <0.0001 |
| | α-DR5 | | | | post-hoc Dunnett | | | | |
| | o42.1 | 150 | nM | 3 | 1way ANOVA with | PBS | 0.05 | **** | <0.0001 |
| | α-DR5 | | | | post-hoc Dunnett | | | | |
| | i52.3 | 150 | nM | 3 | 1way ANOVA with | PBS | 0.05 | ** | 0.0079 |
| | α-DR5 | | | | post-hoc Dunnett | | | | |
| Viability 4d | PBS | 150 | nM | 3 | 1way ANOVA with | N/A | N/A | N/A | N/A |
| RCC4 Fc | | | | | post-hoc Dunnett | | | | |
| cages (4d) | d2.4 Fc | 150 | nM | 3 | 1way ANOVA with | PBS | 0.05 | ns | 0.7157 |
| | | | | | post-hoc Dunnett | | | | |
| | t32.4 Fc | 150 | nM | 3 | 1way ANOVA with | PBS | 0.05 | ns | 0.9976 |
| | | | | | post-hoc Dunnett | | | | |
| | t32.8 Fc | 150 | nM | 3 | 1way ANOVA with | PBS | 0.05 | ns | 0.8556 |
| | | | | | post-hoc Dunnett | | | | |
| | o42.1 Fc | 150 | nM | 3 | 1way ANOVA with | PBS | 0.05 | ns | 0.2309 |
| | | | | | post-hoc Dunnett | | | | |
| | i52.3 Fc | 150 | nM | 3 | 1way ANOVA with | PBS | 0.05 | ns | 0.9302 |
| | | | | | post-hoc Dunnett | | | | |
| Viability 6d | PBS | 150 | nM | 6 | 1way ANOVA with | N/A | N/A | N/A | N/A |
| RCC4 (4e) | | | | | post-hoc Dunnett | | | | |
| | TRAIL | 150 | nM | 6 | 1way ANOVA with | PBS | 0.05 | ns | 0.9996 |
| | | | | | post-hoc Dunnett | | | | |
| | α-DR5 | 150 | nM | 6 | 1way ANOVA with | PBS | 0.05 | ns | >0.9999 |
| | | | | | post-hoc Dunnett | | | | |
| | t32.4 Fc | 150 | nM | 3 | 1way ANOVA with | PBS | 0.05 | ns | 0.9591 |
| | | | | | post-hoc Dunnett | | | | |
| | o42.1 Fc | 150 | nM | 3 | 1way ANOVA with | PBS | 0.05 | ns | 0.9593 |
| | | | | | post-hoc Dunnett | | | | |
| | t32.4 | 150 | nM | 6 | 1way ANOVA with | PBS | 0.05 | **** | <0.0001 |
| | α-DR5 | | | | post-hoc Dunnett | | | | |
| | o42.1 | 150 | nM | 6 | 1way ANOVA with | PBS | 0.05 | *** | 0.0001 |
| | α-DR5 | | | | post-hoc Dunnett | | | | |
| c-PARP | PBS | 150 | nM | 4 | 1way ANOVA with | N/A | N/A | N/A | N/A |
| quant. (4g) | | | | | post-hoc Dunnett | | | | |
| | TRAIL | 150 | nM | 4 | 1way ANOVA with | PBS | 0.05 | ns | 0.0845 |
| | | | | | post-hoc Dunnett | | | | |
| | α-DR5 | 150 | nM | 3 | 1way ANOVA with | PBS | 0.05 | ns | 0.4746 |
| | | | | | post-hoc Dunnett | | | | |
| | o42.1 Fc | 150 | nM | 3 | 1way ANOVA with | PBS | 0.05 | ns | 0.9979 |
| | | | | | post-hoc Dunnett | | | | |
| | o42.1 | 150 | nM | 3 | 1way ANOVA with | PBS | 0.05 | **** | <0.0001 |
| | α-DR5 | | | | post-hoc Dunnett | | | | |
| Caspase-3,7 | PBS | 15 | nM | 2 | 2way ANOVA with | N/A | N/A | N/A | N/A |
| Colo205 | | | | | post-hoc Dunnett | | | | |
| | | 1.5 | nM | 2 | 2way ANOVA with | 15 pM PBS | 0.05 | ns | >0.9999 |
| | | | | | post-hoc Dunnett | | | | |
| | | 150 | nM | 2 | 2way ANOVA with | 15 pM PBS | 0.05 | ns | >0.9999 |
| | | | | | post-hoc Dunnett | | | | |
| | TRAIL | 15 | nM | 2 | 2way ANOVA with | 15 pM PBS | 0.05 | ns | >0.9999 |
| | | | | | post-hoc Dunnett | | | | |
| | | 1.5 | nM | 2 | 2way ANOVA with | 15 pM PBS | 0.05 | *** | 0.0006 |
| | | | | | post-hoc Dunnett | | | | |
| | | 150 | nM | 2 | 2way ANOVA with | 15 pM PAS | 0.05 | **** | <0.0001 |
| | | | | | post-hoc Dunnett | | | | |
| | α-DR5 | 15 | pM | 2 | 2way ANOVA with | 15 pM PBS | 0.05 | ns | 0.9997 |
| | | | | | post-hoc Dunnett | | | | |
| | | 1.5 | nM | 2 | 2way ANOVA with | 15 pM PBS | 0.05 | ns | >0.9999 |
| | | | | | post-hoc Dunnett | | | | |
| | | 150 | nM | 2 | 2way ANOVA with | 15 pM PBS | 0.05 | ns | 0.9996 |
| | | | | | post-hoc Dunnett | | | | |
| | d2.4 | 15 | pM | 2 | 2way ANOVA with | 15 pM PBS | 0.05 | ns | 0.9996 |
| | α-DR5 | | | | post-hoc Dunnett | | | | |
| | | 1.5 | nM | 2 | 2way ANOVA with | 15 pM PBS | 0.05 | **** | <0.0001 |
| | | | | | post-hoc Dunnett | | | | |
| | | 150 | nM | 2 | 2way ANOVA with | 15 pM PBS | 0.05 | **** | <0.0001 |
| | | | | | post-hoc Dunnett | | | | |
| | t32.4 | 15 | pM | 2 | 2way ANOVA with | 15 pM PBS | 0.05 | ns | 0.1074 |
| | α-DR5 | | | | post-hoc Dunnet | | | | |
| | | 1.5 | nM | 2 | 2way ANOVA with | 15 pM PBS | 0.05 | **** | <0.0001 |
| | | | | | post-hoc Dunnett | | | | |
| | | 150 | nM | 2 | 2way ANOVA with | 15 pM PBS | 0.05 | **** | <0.0001 |
| | | | | | post-hoc Dunnett | | | | |
| | t32.8 | 15 | pM | 2 | 2way ANOVA with | 15 pM PBS | 0.05 | ns | >0.9999 |
| | α-DR5 | | | | post-hoc Dunnett | | | | |
| | | 1.5 | nM | 2 | 2way ANOVA with | 15 pM PBS | 0.05 | **** | <0.0001 |
| | | | | | post-hoc Dunnett | | | | |
| | | 150 | nM | 2 | 2way ANOVA with | 15 pM PBS | 0.05 | **** | <0.0001 |
| | | | | | post-hoc Dunnett | | | | |
| | o42.1 | 15 | pM | 2 | 2way ANOVA with | 15 pM PBS | 0.05 | ns | 0.6538 |
| | α-DR5 | | | | post-hoc Dunnett | | | | |
| | | 1.5 | nM | 2 | 2way ANOVA with | 15 pM PBS | 0.05 | **** | <0.0001 |
| | | | | | post-hoc Dunnett | | | | |
| | | 150 | nM | 2 | 2way ANOVA with | 15 pM PBS | 0.05 | **** | <0.0001 |
| | | | | | post-hoc Dunnett | | | | |
| | i52.3 | 15 | pM | 2 | 2way ANOVA with | 15 pM PBS | 0.05 | ns | >0.9999 |
| | α-DR5 | | | | post-hoc Dunnett | | | | |
| | | 1.5 | nM | 2 | 2way ANOVA with | 15 pM PBS | 0.05 | **** | <0.0001 |
| | | | | | post-hoc Dunnett | | | | |
| | | 150 | nM | 2 | 2way ANOVA with | 15 pM PBS | 0.05 | **** | <0.0001 |
| | | | | | post-hoc Dunnett | | | | |
| Caspase-3,7 | PBS | 150 | nM | 3 | 1way ANOVA with | N/A | N/A | N/A | N/A |
| RCC4 Fc | | | | | post-hoc Dunnett | | | | |
| | d2.4 Fc | 150 | nM | 6 | 1way ANOVA with | PBS | 0.05 | * | 0.0129 |
| | | | | | post-hoc Dunnett | | | | |
| | t32.4 Fc | 150 | nM | 6 | 1way ANOVA with | PBS | 0.05 | * | 0.046 |
| | | | | | post-hoc Dunnett | | | | |
| | t32.8 Fc | 150 | nM | 3 | 1way ANOVA with | PBS | 0.05 | ns | 0.9198 |
| | | | | | post-hoc Dunnett | | | | |
| | o42.1 Fc | 150 | nM | 6 | 1way ANOVA with | PBS | 0.05 | ns | 0.2112 |
| | | | | | post-hoc Dunnett | | | | |
| | i52.3 Fc | 150 | nM | 3 | 1way ANOVA with | PBS | 0.05 | ns | 0.9996 |
| | | | | | post-hoc Dunnett | | | | |
| RAM009 | PBS | 1.5 | nM | 6 | 2way ANOVA with | PBS | 0.05 | N/A | N/A |
| Caspase-3,7 | | | | | post-hoc Dunnett | | | | |
| | | 150 | nM | 6 | 2way ANOVA with | PBS | 0.05 | ns | 0.3848 |
| | | | | | post-hoc Dunnett | | | | |
| | TRAIL | 1.5 | nM | 6 | 2way ANOVA with | PBS | 0.05 | ns | 0.9726 |
| | | | | | post-hoc Dunnett | | | | |
| | | 150 | nM | 6 | 2way ANOVA with | PBS | 0.05 | ns | 0.0525 |
| | | | | | post-hoc Dunnett | | | | |
| | α-DR5 | 1.5 | nM | 6 | 2way ANOVA with | PBS | 0.05 | ns | 0.9566 |
| | | | | | post-hoc Dunnett | | | | |
| | | 150 | nM | 6 | 2way ANOVA with | PBS | 0.05 | ns | 0.2752 |
| | | | | | post-hoc Dunnett | | | | |
| | d2.4 | 1.5 | nM | 6 | 2way ANOVA with | PBS | 0.05 | ns | 0.9677 |
| | α-DR5 | | | | post-hoc Dunnett | | | | |
| | | 150 | nM | 6 | 2way ANOVA with | PBS | 0.05 | ** | 0.0076 |
| | | | | | post-hoc Dunnett | | | | |
| | t32.4 | 1.5 | nM | 6 | 2way ANOVA with | PBS | 0.05 | ns | 0.9703 |
| | α-DR5 | | | | post-hoc Dunnett | | | | |
| | | 150 | nM | 6 | 2way ANOVA with | PBS | 0.05 | ** | 0.0028 |
| | | | | | post-hoc Dunnett | | | | |
| | t32.8 | 1.5 | nM | 6 | 2way ANOVA with | PBS | 0.05 | ns | 0.9996 |
| | α-DR5 | | | | post-hoc Dunnett | | | | |
| | | 150 | nM | 6 | 2way ANOVA with | PBS | 0.05 | ** | 0.0067 |
| | | | | | post-hoc Dunnett | | | | |
| | o42.1 | 1.5 | nM | 6 | 2way ANOVA with | PBS | 0.05 | ns | 0.9991 |
| | α-DR5 | | | | post-hoc Dunnett | | | | |
| | | 150 | nM | 6 | 2way ANOVA with | PBS | 0.05 | ** | 0.0038 |
| | | | | | post-hoc Dunnett | | | | |
| | i52.3 | 1.5 | nM | 6 | 2way ANOVA with | PBS | 0.05 | ns | 0.9994 |
| | α-DR5 | | | | post-hoc Dunnett | | | | |
| | | 150 | nM | 6 | 2way ANOVA with | PBS | 0.05 | *** | 0.0006 |
| | | | | | post-hoc Dunnett | | | | |
| | d2.4 Fc | 150 | nM | 6 | 2way ANOVA with | PBS | 0.05 | ns | 0.9966 |
| | | | | | post-hoc Dunnett | | | | |
| | t32.4 Fc | 150 | nM | 6 | 2way ANOVA with | PBS | 0.05 | ns | 0.9997 |
| | | | | | post-hoc Dunnett | | | | |
| | t32.8 Fc | 150 | nM | 6 | 2way ANOVA with | PBS | 0.05 | ns | 0.9992 |
| | | | | | post-hoc Dunnett | | | | |
| | o42.1 Fc | 150 | nM | 6 | 2way ANOVA with | PBS | 0.05 | ns | 0.9994 |
| | | | | | post-hoc Dunnett | | | | |
| | i52.3 Fc | 150 | nM | 6 | 2way ANOVA with | PBS | 0.05 | ns | 0.9995 |
| | | | | | post-hoc Dunnett | | | | |
| RAM009 | PBS | 150 | nM | 6 | 2way ANOVA with | PBS | 0.05 | | |
| Viability | | | | | post-hoc Dunnett | | | | |
| | TRAIL | 150 | nM | 3 | 2way ANOVA with | PBS | 0.05 | ns | 0.9901 |
| | | | | | post-hoc Dunnett | | | | |
| | α-DR5 | 150 | nM | 3 | 2way ANOVA with | PBS | 0.05 | ns | 0.9995 |
| | | | | | post-hoc Dunnett | | | | |
| | d2.4 | 150 | nM | 3 | 2way ANOVA with | PBS | 0.05 | ns | 0.9996 |
| | α-DR5 | | | | post-hoc Dunnett | | | | |
| | t32.4 | 150 | nM | 3 | 2way ANOVA with | PBS | 0.05 | ns | 0.9212 |
| | α-DR5 | | | | post-hoc Dunnett | | | | |
| | t32.8 | 150 | nM | 3 | 2way ANOVA with | PBS | 0.05 | ns | 0.7875 |
| | α-DR5 | | | | post-hoc Dunnett | | | | |
| | o42.1 | 150 | nM | 3 | 2way ANOVA with | PBS | 0.05 | ns | 0.7485 |
| | α-DR5 | | | | post-hoc Dunnett | | | | |
| | i52.3 | 150 | nM | 3 | 2way ANOVA with | PBS | 0.05 | ns | 0.1419 |
| | α-DR5 | | | | post-hoc Dunnett | | | | |
| | d2.4 Fc | 150 | nM | 3 | 2way ANOVA with | PBS | 0.05 | ns | 0.9996 |
| | | | | | post-hoc Dunnett | | | | |
| | t32.4 Fc | 150 | nM | 3 | 2way ANOVA with | PBS | 0.05 | ns | 0.9718 |
| | | | | | post-hoc Dunnett | | | | |
| | t32.8 Fe | 150 | nM | 3 | 2Way ANOVA with | PBS | 0.05 | ns | 0.999 |
| | | | | | post-hoc Dunnett | | | | |
| | o42.1 Fc | 150 | nM | 3 | 2way ANOVA with | PBS | 0.05 | ns | 0.9913 |
| | | | | | post-hoc Dunnett | | | | |
| | i52.3 Fc | 150 | nM | 3 | 2way ANOVA with | PBS | 0.05 | ns | 0.9837 |
| | | | | | post-hoc Dunnett | | | | |

### Titer Glo Cell Viability Assay (4 Day Viability)

Cells were plated onto a 96-well plate at 20,000 cells/well. The next day, cells were treated with 150 nM of a-DR5 AbCs, rhTRAIL and α-DR5 antibody for 4 days. At day 4, 100 μL of CellTiter-Glo reagent (Promega Corp. USA, #G7570) was added to the 100 μL of media per well, incubated for 10 min at 37° C. and luminescence was measured using a Perkin-Elmer Envision plate reader.

### Alamar Blue Cell Viability Assay (6 Day Viability)

Cells were seeded onto a 12-well tissue culture plate at 50,000 cells/well. The next day, cells were treated with α-DR5 AbCs, rhTRAIL, or α-DR5 antibodies at 150 nM concentration. Three days later, cells were passaged at 30,000 cells/well and treated with 150 nM of α-DR5 cages, rhTRAIL and α-DR5 antibody for 3 days. At 6 days, the media was replaced with 450 μL/well of fresh media and 50 μL of Alamar™ blue reagent (Thermofisher Scientific, USA, #DAL1025) was then added. After 4 hours of incubation at 37° C., 50 μL of media was transferred into a 96-well opaque white plate and fluorescence intensity was measured using plate reader according to manufacturer's instructions.

### Protein Analysis

Cells were passaged onto a 12-well plate at 40,000 cells/well and were grown until 80% confluency is reached. Before treatment, the media was replaced with 500 μL of fresh media. For DR5 experiments, AMG-655 antibody and rhTRAIL were added at 150 nM concentration and Fc-only nanocages or α-DR5 nanocages were added at 150 nM, 1.5 nM and 15 pM concentration onto the media and incubated for 24 hours at 37° C. prior to protein isolation.

Media containing dead cells was transferred to a 1.5 ml Eppendorf tube, and the cells were gently rinsed with 1× phosphate buffered saline. 1× trypsin was added to the cells for 3 min. All the cells were collected into the 1.5 mL Eppendorf containing the medium with dead cells. Cells were washed once in PBS 1× and lysed with 70 μL of lysis buffer containing 20 mM Tris-HCl (pH 7.5), 150 mM NaCl, 15% Glycerol, 1% Triton, 3% SDS, 25 mM β-glycerophosphate, 50 mM NaF, 10 mM Sodium Pyrophosphate, 0.5% Orthovanadate, 1% PMSF (all chemicals were from Sigma-Aldrich, St. Louis, MO), 25 U Benzonase Nuclease (EMD Chemicals, Gibbstown, NJ), protease inhibitor cocktail (Pierce™ Protease Inhibitor Mini Tablets, Thermo Scientific, USA), and phosphatase inhibitor cocktail 2 (catalog #P5726), in their respective tubes. Total protein samples were then treated with 1 μL of Benzonase (Novagen, USA) and incubated at 37° C. for 10 min. 21.6 μL of 4× Laemmli Sample buffer (Bio-Rad, USA) containing 10% beta-mercaptoethanol was added to the cell lysate and then heated at 95° C. for 10 minutes. The boiled samples were either used for Western blot analysis or stored at −80° C.

### Production of A1F-Fc

Synthetic genes were optimized for mammalian expression and subcloned into the CMV/R vector (VRC 8400; PMID: 15994776). XbaI and AvrII restriction sites were used for insertion of A1F-Fc. Gene synthesis and cloning was performed by Genscript. Expi 293F cells were grown in suspension using Expi293 Expression Medium (Thermo Fisher Scientific) at 150 RPM, 5% CO₂, 70% humidity, 37° C. At confluency of ˜2.5×10⁶ cells/mL.

the cells were transfected with the vector encoding A1F-Fc (1000 μg per 1 L of cells) using PEI MAX (Polysciences) as a transfection reagent. Cells were incubated for 96 hours, after which they were spun down by centrifugation (4,000×g, 10 min, 4° C.) and the protein-containing supernatant was further clarified by vacuum-filtration (0.45 μm, Millipore Sigma). In preparation of nickel-affinity chromatography steps, 50 mM Tris, 350 mM NaCl, pH 8.0 was added to clarified supernatant. For each liter of supernatant, 4 mL of Ni Sepharose™ excel resin (GE) was added to the supernatant, followed by overnight shaking at 4° C. After 16-24 hours, resin was collected and separated from the mixture and washed twice with 50 mM Tris, 500 mM NaCl, 30 mM imidazole, pH 8.0 prior to elution of desired protein with 50 mM Tris, 500 mM NaCl, 300 mM imidazole, pH 8.0. Eluates were purified by SEC using a Superdex™ 200 Increase column.

### Western Blotting

The protein samples were thawed and heated at 95° C. for 10 minutes. 10 μL of protein sample per well was loaded and separated on a 4-10% SDS-PAGE gel for 30 minutes at 250 Volt. The proteins were then transferred onto a Nitrocellulose membrane for 12 minutes using the semi-dry turbo transfer western blot apparatus (Bio-Rad, USA). Post-transfer, the membrane was blocked in 5% nonfat dry milk for 1 hour. After 1 hour, the membrane was probed with the respective antibodies: cleaved-PARP (Cell Signaling, USA) at 1:2000 dilution; cFLIP (R&D systems, USA) at 1:1000 dilution; pERK1/2 (Cell Signaling) at 1:5000 dilution; pFAK (Cell Signaling) at 1:1000 dilution; p-AKT (S473) (Cell Signaling) at 1:2000 dilution; and actin (Cell Signaling, USA) at 1:10,000 dilution. Separately, for p-AKT (S473) the membrane was blocked in 5% BSA for 3 hours followed by primary antibody addition. Membranes with primary antibodies were incubated on a rocker at 4° C., overnight. Next day, the membranes were washed with 1× TBST (3 times, 10 minutes interval) and the respective HRP-conjugated secondary antibody (Bio-Rad, USA) (1:10,000) was added and incubated at room temperature for 1 hour. For p-AKT(S473), following washes, the membrane was blocked in 5% milk at room temperature for 1 hour and then incubated in the respective HRP-conjugated secondary antibody (1:2000) prepared in 5% milk for 2 hours. After secondary antibody incubation, all the membranes were washed with 1× TBST (3 times, 10 minutes interval) and developed using Luminol reagent and imaged using Bio-Rad ChemiDoc™ Imager. Data were quantified using the ImageJ™ software to analyze band intensity. Quantifications were done by calculating the peak area for each band. Each signal was normalized to the actin quantification from that lane of the same gel, to allow for cross-gel comparisons. Fold-changes were then calculated compared to PBS for all samples except for the pAKT reported for the A1F-Fc western blot (there was not enough pAKT signal for comparison, so o42.1 A1F-Fc was used for normalization). Statistical comparisons were performed using Graphpad Prism™ (see Tables 11 and 12 for full detail).

TABLE 12
Statistical information for A1F-Fe experiments.

| Experiment | | | | Mean | | | Adjusted |
|---|---|---|---|---|---|---|---|
| (FIG.) | Condition | n | Test | compared to | α | Summary | P value |
| pAKT (4j, | PBS | 13 | 1way ANOVA with | N/A | N/A | N/A | N/A |
| 9c (H8)) | | | post-hoc Dunnett | | | | |
| | A1F-Fc | 3 | 1way ANOVA with | PBS | 0.05 | ns | >0.9999 |
| | | | post-hoc Dunnett | | | | |
| | o42.1 Fc | 4 | 1way ANOVA with | PBS | 0.05 | ns | >0.9999 |
| | | | post-hoc Dunnett | | | | |
| | i52.3 Fc | 3 | 1way ANOVA with | PBS | 0.05 | ns | >0.9999 |
| | | | post-hoc Dunnett | | | | |
| | o42.1 | 9 | 1way ANOVA with | PBS | 0.05 | **** | <0.0001 |
| | A1F-Fc | | post-hoc Dunnett | | | | |
| | i52.3 | 8 | 1way ANOVA with | PBS | 0.05 | **** | <0.0001 |
| | A1F-Fc | | post-hoc Dunnett | | | | |
| | H8-A1F | 4 | 1way ANOVA with | PBS | 0.05 | **** | <0.0001 |
| | | | post-hoc Dunnett | | | | |
| pERK1-2 | PBS | 13 | 1way ANOVA with | N/A | N/A | N/A | N/A |
| (4j; 9c (H8)) | | | post-hoc Dunnett | | | | |
| | A1F-Fc | 3 | 1way ANOVA with | PBS | 0.05 | ns | 0.9997 |
| | | | post-hoc Dunnett | | | | |
| | o42.1 Fc | 4 | 1way ANOVA with | PBS | 0.05 | ns | 0.9957 |
| | | | post-hoc Dunnett | | | | |
| | i52.3 Fc | 3 | 1way ANOVA with | PBS | 0.05 | ns | 0.9997 |
| | | | post-hoc Dunnett | | | | |
| | o42.1 | 9 | 1way ANOVA with | PBS | 0.05 | ** | 0.0032 |
| | A1F-Fc | | post-hoc Dunnett | | | | |
| | i52.3 | 8 | 1way ANOVA with | PBS | 0.05 | **** | <0.0001 |
| | A1F-Fc | | post-hoc Dunnett | | | | |
| | H8-A1F | 6 | 1way ANOVA with | PBS | 0.05 | * | 0.0112 |
| | | | post-hoc Dunnett | | | | |
| Vascular | PBS | 7 | 1way ANOVA with | N/A | N/A | N/A | N/A |
| stability (4k; | | | post-hoc Dunnett | | | | |
| 9c (H8)) | A1F-Fc | 6 | 1way ANOVA with | PBS | 0.05 | ns | 0.9932 |
| | | | post-hoc Dunnett | | | | |
| | o42.1 Fc | 4 | 1way ANOVA with | PBS | 0.05 | ns | >0.9999 |
| | | | post-hoc Dunnett | | | | |
| | i52.3 Fc | 3 | 1way ANOVA with | PBS | 0.05 | ns | 0.8699 |
| | | | post-hoc Dunnett | | | | |
| | o42.1 | 6 | 1way ANOVA with | PBS | 0.05 | *** | 0.0006 |
| | A1F-Fc | | post-hoc Dunnett | | | | |
| | i52.3 | 5 | 1way ANOVA with | PBS | 0.05 | **** | <0.0001 |
| | A1F-Fc | | post-hoc Dunnett | | | | |
| | H8-A1F | 4 | 1way ANOVA with | PBS | 0.05 | * | 0.0208 |
| | | | post-hoc Dunnett | | | | |
| pAKT (HS | PBS | 3 | 1way ANOVA with | o42.1 | 0.05 | *** | <0.0001 |
| exp.) | | | post-hoc Donnett | A1F-Fc | | | |
| | o42.1 | 3 | 1way ANOVA with | o42.1 | 0.05 | N/A | N/A |
| | A1F-Fc | | post-hoc Dunnett | A1F-Fc | | | |
| | 10% HS | 3 | 1way ANOVA with | o42.1 | 0.05 | *** | 0.0002 |
| | | | post-hoc Dunnett | A1F-Fc | | | |
| | o42.1 | 3 | 1way ANOVA with | o42.1 | 0.05 | ns | 0.9431 |
| | A1F-Fc | | post-hoc Donnett | A1F-Fc | | | |
| | 10% HS | | | | | | |
| | 4° C. | | | | | | |
| | o42.1 | 3 | 1way ANOVA with | 042.1 | 0.05 | ns | 0.9998 |
| | A1F-Fc | | post-hoc Dunnett | A1F-Fc | | | |
| | 10% HS | | | | | | |
| | 37° C. | | | | | | |

### Tube Formation Assay (Vascular Stability)

Tube formation was done with modified protocol from Liang et al., 2007. Briefly, passage 6 HUVECs were seeded onto 24-well plates precoated with 150 μL of 100% cold Matrigel™ (Corning, USA) at 150,000 cells/well density along with scaffolds at 89 nM A1F-Fc concentrations or PBS in low glucose DMEM medium supplemented with 0.5% FBS for 24 hours. At the 24 hour time point, old media is aspirated and replaced with fresh media without scaffolds. The cells continue to be incubated up to 72 hours. Cells were imaged at 48-hour and 72-hour time points using Leica Microscope at 10× magnification under phase contrast. Thereafter, the tubular formations were quantified by calculating the number of nodes, meshes and tubes using Angiogenesis Analyzer plugin in Image J software. Vascular stability is calculated by averaging the number of nodes, meshes, and tubes then normalizing to PBS. Statistical comparisons were performed using Graphpad Prism™ (see Table 12 for full detail).

### Immune Cell Activation Materials and Methods

### CD40 Luminescence Assay

A non-agonistic antibody (clone LOB7/6, product code MCA1590T, BioRad), was combined with the octahedral o42.1 AbC-forming design as described above and the AbCs were characterized by DLS and NS-EM (FIG. 5). Negative control o42.1 AbC was made using a non-CD40 binding IgG (mpe8), which binds to RSV spike protein (45). These two AbCs, along with uncaged LOB7/6 and a positive control CD40-activating IgG (Promega, catalog #K118A) were diluted to make a 10-point, threefold dilution series for triplicate technical repeats starting at 1.2 μM. The positive control CD40-activating IgG (K118A) is a murine IgG1a antibody, and so it was not compatible for assembly with the o42.1 design, likely due to the low binding interface between protein A and mIgG1a (data not shown).

To assay CD40 activation, we followed manufacturer's instructions for a bioluminescent cell-based assay that measures the potency of CD40 response to external stimuli such as IgGs (Promega, JA2151). Briefly, CD40 effector Chinese Hamster Ovary (CHO) cells were cultured and reagents were prepared according to the assay protocol. The antibodies and AbCs were incubated with the CD40 effector CHO cells for 8 hours at 37° C., 5% CO2. Bio-Glo™ Luciferase Assay System (G7941) included in the assay kit was used to visualize the activation of CD40 from luminescence readout from a plate reader. The Bio-Glo™ Reagent was applied to the cells and luminescence was detected by a Synergy Neo2 plate reader every min for 30 minutes. Data were analyzed by averaging luminescence between replicates and subtracting plate background. The fold induction of CD40-binding response was determined by RLU of sample normalized to RLU of no antibody controls. Data curves were plotted and EC50 was calculated using GraphPad Prism™ using the log(agonist) vs. response—Variable slope (four parameters); see Table 7 for EC50 values and 95% CI values.

We claim:

1. An Antibody Cage (AbC) nanoparticle, comprising:
(a) a plurality of antibodies, each antibody comprising at least two fragment crystallizable (Fc) domains, and
(b) a plurality of cyclic oligomers, each cyclic oligomer comprising a polypeptide comprising:
(i) an Fc-binding domain,
(ii) a monomer domain to position the Fc-binding domains to promote assembly of the nanoparticle, and
(iii) an oligomer domain to assemble the polypeptides to form the cyclic oligomer.
wherein the Fc domains of the plurality of antibodies non-covalently interact with Fc-binding domains of the plurality of cyclic oligomers to form the nanoparticle.

2. The AbC nanoparticle of claim 1, wherein the Fc domains of the antibodies dimerize to form a structural component of the nanoparticle.

3. The AbC nanoparticle of claim 1, wherein the nanoparticle displays antigen-binding domains of the antibodies on an exterior of the nanoparticle.

4. The AbC nanoparticle of claim 1, wherein the monomer domain comprises a helical domain.

5. The AbC nanoparticle of claim 1, wherein the oligomer domain comprises a polypeptide sequence at least 90% identical to residues 168-354 of SEQ ID NO: 1, residues 172-354 of SEQ ID NO: 2, or residues 184-371 of SEQ ID NO: 3.

6. The AbC nanoparticle of claim 1, wherein the oligomer domain comprises a polypeptide sequence at least 90% identical to residues 163-353 of SEQ ID NO: 4, residues 163-351 of SEQ ID NO: 5, residues 138-304 of SEQ ID NO: 6, or residues 257-412 of SEQ ID NO: 7.

7. The AbC nanoparticle of claim 1, wherein the oligomer domain comprises a polypeptide sequence at least 90% identical to residues 290-345 of SEQ ID NO: 8 or residues 293-354 of SEQ ID NO: 9.

8. The AbC nanoparticle of claim 5, wherein the Fc-binding domain comprises a polypeptide sequence at least 90% identical to residues 2-100 of SEQ ID NO: 1, residues 2-100 of SEQ ID NO: 2, residues 2-104 of SEQ ID NO: 3.

9. The AbC nanoparticle of claim 6, wherein the Fc-binding domain comprises a polypeptide sequence at least 90% identical to residues 2-47 of SEQ ID NO: 4, residues 2-47 of SEQ ID NO: 5, residues 2-39 of SEQ ID NO: 6, or residues 2-46 of SEQ ID NO: 7.

10. The AbC nanoparticle of claim 7, wherein the Fc-binding domain comprises a polypeptide sequence at least 90% identical to residues 2-119 of SEQ ID NO: 8, or residues 2-135 of SEQ ID NO: 9.

11. The AbC nanoparticle of claim 8, wherein the monomer domain comprises a polypeptide sequence at least 90% identical to residues 101-167 of SEQ ID NO: 1 or residues 101-171 of SEQ ID NO: 2.

12. The AbC nanoparticle of claim 8, wherein the monomer domain comprises a polypeptide sequence at least 90% identical to residues 105-183 of SEQ ID NO: 3.

13. The AbC nanoparticle of claim 9, wherein the monomer domain comprises a polypeptide sequence at least 90% identical to residues 48-162 of SEQ ID NO: 4 or residues 48-162 of SEQ ID NO: 5.

14. The AbC nanoparticle of claim 9, wherein the monomer domain comprises a polypeptide sequence at least 90% identical to residues 40-137 of SEQ ID NO: 6.

15. The AbC nanoparticle of claim 9, wherein the monomer domain comprises a polypeptide sequence at least 90% identical to residues 47-256 of SEQ ID NO: 7.

16. The AbC nanoparticle of claim 10, wherein the monomer domain comprises a polypeptide sequence at least 90% identical to residues 120-289 of SEQ ID NO: 8 or residues 136-292 of SEQ ID NO: 9.

17. The AbC nanoparticle of claim 1, wherein the polypeptide comprises a polypeptide sequence according to residues 2-354 of SEQ ID NO: 1.

18. The AbC nanoparticle of claim 1, wherein the polypeptide comprises a polypeptide sequence according to residues 2-354 of SEQ ID NO: 2.

19. The AbC nanoparticle of claim 1, wherein the polypeptide comprises a polypeptide sequence according to residues 2-371 of SEQ ID NO: 3.

20. The AbC nanoparticle of claim 1, wherein the polypeptide comprises a polypeptide sequence according to residues 2-353 of SEQ ID NO: 4.

21. The AbC nanoparticle of claim 1, wherein the polypeptide comprises a polypeptide sequence according to residues 2-351 of SEQ ID NO: 5.

22. The AbC nanoparticle of claim 1, wherein the polypeptide comprises a polypeptide sequence according to residues 2-304 of SEQ ID NO: 6.

23. The AbC nanoparticle of claim 1, wherein the polypeptide comprises a polypeptide sequence according to residues 2-412 of SEQ ID NO: 7.

24. The AbC nanoparticle of claim 1, wherein the polypeptide comprises a polypeptide sequence according to residues 2-345 of SEQ ID NO: 8.

25. The AbC nanoparticle of claim 1, wherein the polypeptide comprises a polypeptide sequence according to residues 2-354 of SEQ ID NO: 9.

26. A pharmaceutical composition comprising the AbC nanoparticle of claim 1.

27. A method of making an Antibody Cage (AbC) nanoparticle according to claim 1, comprising contacting the plurality of antibodies with the plurality of cyclic oligomers under conditions suitable to promote assembly of the nanoparticle.

28. A polypeptide comprising:
(i) an Fc-binding domain,
(ii) a monomer domain to position the Fc-binding domains to promote assembly of the nanoparticle, and
(iii) an oligomer domain to assemble the polypeptides to form the cyclic oligomer.

29. A polynucleotide encoding the polypeptide of claim 28.

30. A host cell configured to express the polypeptide of claim 28.
